(19) United States
(12) Patent Application Publication
(10) Pub. No.: US 2025/0354168 A1
(43) Pub. Date: Nov. 20, 2025

(54) NOVEL AAV CAPSID-MODIFIED STRAIN AND USE THEREOF

(71) Applicant: SHANGHAI LANGSHENG BIOTECHNOLOGY CO., LTD, Shanghai (CN)

(72) Inventors: Fei WU, Shanghai (CN); Zhuoyu NI, Shanghai (CN)

(21) Appl. No.: 18/859,925

(22) PCT Filed: Apr. 21, 2023
(86) PCT No.: PCT/CN2023/089805

§ 371 Date: Oct. 24, 2024

(30) Foreign Application Priority Data
Apr. 24, 2022 (CN) 202210436003.4

(51) Int. Cl. C12N 15/86; A61K 48/00; C07K 14/005

(52) U.S. Cl. CPC C12N 15/86; A61K 48/0041; A61K 48/0075; C07K 14/005; C12N 2750/14122; C12N 2750/14143; C12N 2750/14152

(57) ABSTRACT

Provided are a method for engineering rAAV vectors to improve the retina tissue tropism, and infection and expression capability thereof and reduce the immunogenicity thereof, a vector obtained by the method, and the use thereof.

The present invention relates to the field of gene therapy. Particularly, the present invention relates to a method for engineering AAV vectors to improve the retinal tissue-tropism and the capability of infection and expression, as well as a vector obtained by the method and the use thereof.

### BACKGROUND ART

Currently the number of people blinded due to inherited retinal diseases (IRDs) is about fifteen million worldwide, accounting for about 0.02% of total population. Varieties of the inherited retinal disease, for example, retinoschisis, etc., are numerous, and more than 200 of the disease-causing genes have been identified so far.

AAV vector is currently one of gene therapy vectors with the most promising application because of its little pathogenicity and loss of the ability to integrate into the genome of the infected cell; and as compared to many types of vectors, the AAV vector also involve lower immunogenicity due to its low pathogenicity. Currently, there are several of AAV vector-based gene therapy medications put on market in some countries and areas, for example, Glybera (the generic name: alipogene tiparvovec) which is an rAAV (recombinant AAV) product marketed on European in 2012 for treating lipoprotein lipase deficiency, Luxturna which is an rAAV product approved to market in 2017 for treating retina disorders), and Zolgensma (the generic name: Onasemnogene abeparvovec) approved to USA market in 2019 for treating spinal muscular atrophies. For IRD, the AAV vector-based gene therapy medication is likewise very promising therapeutic approach. It is known that AAV types 1, 4, 5, 7, 8, and 9 are all able to transduce retinal pigment epithelium cells or photoreceptor cells through subretinal or topical administration, with the transduction efficiency greatly reduced, however, upon IVT (intravitreal) administration.

There have been various attempts to improve the infection efficiency of AAV viruses for the retinal tissue among the current patented technologies. For example, the Chinese patent CN107012171B is directed to an AAV2.7m8, and it is engineered from a known serotype AAV2 where the amino acid at the position 588 in the AAV2 capsid VP1 protein is inserted by a peptide of 11 amino acids targeting the retinal tissue, resulting in the alteration of the conventional capability of AAV2 viral coat for binding to the HSPG receptor. Nonetheless, in the prior art technologies, intravitreal administration of the high dose of AAV2.7m8 still fails to effectively transduce the retinal tissue, especially the retinal pigment epithelium (RPE) and the photoreceptor cells.

### SUMMARY OF THE INVENTION

The present invention provides a method for improving rAAV vectors, characterized in the modification of the amino acid residues (for example, but not limited to, substitution, deletion and/or addition) on the basis of the wild-type AAV2 sequence, leading to increased affinity of the modified AAV2 vector toward the receptors on the target cell's surface. Therefore in a preferred embodiment, said method is a method for engineering an AAV2 capsid protein. In a more preferred embodiment, the method is to engineer the AAV2 capsid protein VP1 to have the following amino acid mutated sites: Q464V, A467P, D469N, I470M, R471A, D472V, S474G, Y500F, and S501A. In another more preferred embodiment, the method is to engineer the AAV2 capsid protein VP1 to have the following amino acid mutated sites: Q464V, A467P, D469N, I470M, R471A, D472V, S474G, Y444F, Y500F, S501A, and Y730F. In another more preferred embodiment, the method is to engineer the AAV2 capsid protein VP1 to have the following amino acid mutated sites: Q464V, A467P, D469N, I470M, R471A, D472V, S474G, Y500F, and S501A, simultaneously with the amino acid sequence LALGDVTRPA insertion between the sites 587(N) and 588(R).

The present invention further provides novel rAAV vectors. Particularly, the present invention provides adeno-associated virus (AAV) serotypes with engineered and optimized VP1 capsid protein, and the corresponding recombinant adeno-associated virus vectors thereof, characterized in that the engineered VP1 capsid protein has an amino acid sequence set forth in any one of SEQ ID NOs: 1-3.

The present invention further provides novel rAAV vectors improved by the method of the present invention, characterized by engineered VP1 capsid protein, which has an amino acid sequence set forth in any one of SEQ ID NOs: 1-3.

In one embodiment of the present invention, the novel serotype was a variant of AAV2. In some embodiments of the present invention, said capsid-engineered-strain binds to the HSPG receptor. In some other preferred embodiment of the invention, said capsid-engineered-strain does not bind to or substantially does not bind to the HSPG receptor. In one preferred embodiment of the present invention, the novel serotype rAAV vector can be effective in the transduction of retina tissues (especially RPE and photoreceptor cells), with a significantly increased efficiency of transduction. In some embodiments, the novel serotype rAAV vector differs in the receptor-binding property, cell/tissue tropism, and transduction efficiency from the wild-type AAV-based (for example, wild-type AAV2-based) rAAV vectors or other rAAV vectors known in the prior art. In some embodiments, the differences in the receptor-binding property, the cell/tissue tropism, and the transduction efficiency of the novel serotype rAAV vectors and the wild-type AAV-based (for example, wild-type AAV2-based) rAAV vectors, are ascribed to modifications on the amino acid sequence (for example, substitution, deletion and/or addition).

In some embodiments of the present invention, the in vitro transduction efficiency of the novel serotype rAAV vector for the retina tissue is increased by at least 5-fold or at least 10-fold, preferably at least 15-fold, more preferably 20-fold, most preferably at least 30- to 50-fold. In some embodiments of the present invention, the increased transduction efficiency is manifested as the increased proportion of the infected cells and/or an increase in the total expression of the exogenous gene in the infected tissue. In some preferable embodiments of the present invention, the increased transduction efficiency occurs in a detectable manner on Day 1 post infection (pi), Day 2 pi, Day 3 pi, Day 4 pi, Day 5 pi, Day 6 pi, Day 7 pi, Day 8 pi, Day 9 pi, or Day 10 pi. In some preferable embodiments of the present invention, the increased transduction efficiency lasts till Week 1 post infection (pi), Week 2 pi, Week 3 pi, Week 4 pi, Week 5 pi, Week 6 pi, Week 7 pi, Week 8 pi, Month 3 pi, Month 4 pi, Month 5 pi, Month 6 pi, Month 7 pi, Month 8 pi, Month 9 pi, Month 10 pi, Month 11 pi, or Month 12 pi after the infection of the tissue.

In some embodiments, the novel serotype rAAV vector of the present invention also has the ability to effectively permeate into the inner limiting layer of the retina tissue, reach the retinal pigment epithelium (REP) layer, distribute throughout and infect the whole mesh layers of the retinochoroidal membrane even at a low dose, and the ability to permeate into, distribute throughout and infection the whole mesh layers of the retinochoroidal membrane being increased by at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 50-fold, at least 80-fold, at least 100-fold.

In a more preferred embodiment of the invention, the novel serotype rAAV vector of the present invention has the considerably increased resistance to the AAV-neutralizing antibodies. In some more preferred embodiments, the novel serotype rAAV vector of the present invention is capable of tolerating or resisting to the 5-fold or 10-fold higher concentration of the AAV-neutralizing antibodies. In some more preferred embodiments, the novel serotype rAAV vector of the present invention is capable of tolerating or resisting to the 20-fold higher concentration of the AAV-neutralizing antibodies. In some more preferred embodiments, the novel serotype rAAV vector of the present invention is capable of tolerating or resisting to the 30-fold or 50-fold higher concentration of the AAV-neutralizing antibodies.

In some aspects, the invention provides rAAV vectors for gene therapy applications. In some aspects, the present invention further provides a method of delivering the rAAV vectors for gene therapy applications to retinal cells of an individual and a method for treating ocular disorders.

In some aspects, the invention provides an isolated nucleic acid molecule, which encodes an AAV capsid protein having the amino acid sequence set forth in any one of SEQ ID NOs: 1-3. In some embodiments of the present invention, the isolated nucleic acid molecule comprises a sequence selected from the group consisting of SEQ ID NOs: 4-6. In some embodiments of the present invention, the isolated nucleic acid molecule comprises a sequence having 70%, 80%, 90%, 95%, 96%, 97%, 98%, 99%, or more than 99% sequence identity with SEQ ID NOs: 4-6. In some embodiments, fragments of the isolated nucleic acid molecule are provided. In certain embodiments, the fragments of the isolated nucleic acid molecule do not encode a peptide of the amino acid sequence of SEQ ID NO: 8.

In certain aspects of the invention, provided is a composition comprising any of the engineered VP1 capsid proteins as described above. In some embodiments, the composition further comprises pharmaceutically acceptable excipients. In some embodiments, provided is a composition comprising one or more of the VP1 capsid proteins of the present invention and physiologically compatible carriers. In some preferred embodiments, provided is a composition in which the VP1 capsid protein is present in the composition in such a form that the protein exists in the entire virus particles.

In certain aspects of the invention, provided is a rAAV vector comprising the engineered VP1 capsid protein as described above. In some embodiments, provided is a composition comprising the rAAV vector. In certain embodiments, the composition comprising the rAAV vector further comprise pharmaceutically acceptable excipients. Also provided is a rAAV vector which comprise one or more of the isolated AAV capsid proteins of the present invention.

In some aspects of the invention, provided is a host cell comprising an isolated nucleic acid molecule having a sequence selected from the group consisting of SEQ ID NOs: 4-6. In some embodiments, provided is a composition comprising a host cell and cultivation medium. In some embodiments, provided is a composition comprising a host cell and a cryoprotectant.

According to some aspects of the invention, provided is a method of delivering an exogenous gene to an individual. In some embodiments, the method comprises administering any of the rAAV vectors as described above to an individual, wherein said rAAV vector comprises at least one exogenous gene and infects the cells of a target tissue in said individual. In some embodiments, the individual is selected from the group consisting of the mouse, rat, leporid, canine, feline, ovis, porcine and non-human primate. In one embodiment, the individual is a human being. In some embodiments, said at least one exogenous gene is a protein-coding gene. In certain embodiments, the rAAV vector is administered to the individual intravenously, transdermally, intra-ocularly, intra-thecally, intracerebrally, orally, intramuscularly, subcutaneously, intranasally or by inhalation. In certain embodiments, rAAV vector is administered to an individual by eye drop instillation, intra-ocular, subconjunctival, intracameral, intravitreal, or subretinal injection.

In some embodiments, the individual receiving administration of the rAAV vector had received administration of rAAV vectors and/or been infected with AAV. In some embodiments, the individual receiving administration of the rAAV vector has pre-existing immunity to AAV in vivo. In some preferred embodiments, the individual receiving administration of the rAAV vector has AAV-neutralizing antibodies at the neutralization titer, and consequently the wild-type AAV-based rAAV vector or other existing rAAV vectors can't be delivered to and/or infect the target tissue because of being neutralized by the antibodies. In some preferred embodiments, the individual receiving administration of the rAAV vector has AAV-neutralizing antibodies at the neutralization titer, or at the 5-fold neutralization titer, or 10-fold neutralization titer, or 20-fold neutralization titer, or 30-fold neutralization titer, or 50-fold neutralization titer, and so the wild-type AAV-based rAAV vector or other existing rAAV vectors can't be delivered to and/or infect the target tissue because of being neutralized by the neutralizing antibodies.

In some other aspects of the invention, provided is a kit for the producing the rAAV vector of the present invention. In some embodiments, the kit comprises containers which holds the isolated nucleic acids having the sequence of any one of SEQ ID NOs: 4-6. In some embodiments, the kit further comprises the instructions for producing the rAAV. In some embodiments, the kit further comprises at least one container holding the recombinant AAV vector, wherein the recombinant AAV vector comprises an exogenous gene.

In some other aspects, the present invention involves the use of AAV-based vectors for the purpose of gene delivery, therapy, prophylaxis and research. In some aspects, the present invention relates to such a novel AAV serotype that exhibiting unique tissue/cell-type tropism and/or specificity, and the tropism and/or the specificity is preferably retinal tropism and/or specificity, and more preferably RPE and/or photoreceptor cells tropism and/or specificity. In some embodiments, the novel AAV serotype-based vectors attains stable transferring of genes into somatic cells in animal tissues at a level similar to that of adenovirus vectors (for example, up to nearly 100% tissue transduction in vivo, possibly dependent upon the target tissue and the dose of the vector) and brings no or little vector-related toxicity.

In another aspect, the rAAV vector of the present invention can be useful in a method for delivering a transgene to an individual. The method is performed by administering the rAAV vector of the present invention to the individual, wherein the rAAV vector comprises at least one exogenous gene. In some embodiments, the rAAV vectors targets the individual's predefined tissues.

In one embodiment, the rAAV vector comprises an AAV capsid protein VP1 having the amino acid sequence of any one of SEQ ID NOs: 1-3.

In some embodiments, the exogenous gene expresses a reporter, and the reporter is optionally a reporter enzyme (such as beta-galactosidase), a luciferase (such as firefly luciferase) or a fluorescent protein (such as GFP, DsRed and the like).

In one embodiment, the target tissue for the rAAV vector is retina. In some embodiments, the rAAV vectors transduce RPE and/or photoreceptor cells.

In some embodiments, the rAAV is administered at a dose of 10¹⁰, 10¹¹, 10¹², 10¹³, 10¹⁴, or 10¹⁵ genome copies/subject. In some embodiments, the rAAV is administered at a dose of 10¹⁰, 10¹¹, 10¹², 10¹³, or 10¹⁴ genome copies/kilogram of body weight. The rAAV may be administered by any route. For example, it may be administered intravenously in some embodiment and administered by intravitreal injection in some other embodiments.

According to another aspect of the present invention, provided is a kit for producing the rAAV vector of the present invention. The kit comprises at least one container holding the recombinant AAV vector, at least one container holding rAAV-packaging components and the instructions for producing the recombinant AAV vector.

The rAAV vector-packaging components can include a host cell expressing at least one rep gene and/or at least one cap gene. In some embodiments, the host cell expresses at least one rep gene and/or at least one cap gene through exogenous introduction. In some embodiments, the host cell expresses at least one rep gene and/or at least one cap gene through an exogenous gene which has been integrated into an endogenous expression system. In some embodiments, the host cell is the HEK293T cell. In some other embodiments, the host cell expresses at least one gene product of the helper virus which has an influence on generation of rAAVs containing recombinant AAV vectors. Preferably, said at least one cap gene encode the preferred capsid protein of the present invention.

In some other embodiments, the rAAV-packaging components include a helper virus, optionally wherein the helper virus is adenovirus or herpesvirus.

The rAAV vector and the components therein can comprise any element as described herein. For example, in some embodiments, the rAAV vector comprises an exogenous gene.

In some aspects of the present invention, provided is a pharmaceutical composition comprising the aforesaid rAAV vector which has any of the engineered VP1 capsid proteins as described above and pharmaceutical acceptable carriers, thinners, excipients or buffers.

In some other aspects of the invention, provided is such a kit that comprises a container holding a rAAV vector having any of the engineered VP1 capsid proteins as described above. In some embodiments, the container in the kit is an injector.

In some other aspects of the invention, provided is the use of the rAAV vector, the pharmaceutical composition, and/or the kit of the present invention as specified above, in the manufacture of a medication for treating diseases. In some embodiments, the disease is an ocular disorder. In some embodiments, the disease is a retinal disease. In some preferred embodiments, the disease is IRD. In some embodiments, the medication is prepared as suitable for systemic, intravenous, intra-muscular, subcutaneously, oral, topical, topically contacting, intraperitoneal, or intralesional administration. In some preferred embodiments, the medication is prepared suitable for administration by eye drop instillation, intra-ocular injection, conjunctival injection, intracameral injection, intravitreal injection, or subretinal injection. In some embodiments, the medication is used for the treatment of an individual who had been treated with rAAV vectors and/or infected naturally by AAV. In some embodiments, the medication is used for the treatment of an individual having AAV-neutralizing antibodies in the body, which are at the neutralization titer, and consequently the wild-type AAV-based rAAV vector or other existing rAAV vectors can't be delivered to and/or infect the target tissue because of being neutralized by the neutralizing antibodies. In some embodiments, the medication is used for the treatment of an individual having AAV-neutralizing antibodies in the body, which are at the neutralization titer, or at the 5-fold neutralization titer, or 10-fold neutralization titer, or 20-fold neutralization titer, or 30-fold neutralization titer, or 50-fold neutralization titer, and consequently the wild-type AAV-based rAAV vector or other existing rAAV vectors can't be delivered to and/or infect the target tissue due to being neutralized by the neutralizing antibodies.

### BRIEF DESCRIPTION OF THE DRAWINGS

FIG. 1 shows the preparation and caspids analysis of the three serotypes RC-C08, RC-C15, and RC-C18 of the present invention. A-C indicate the plasmid maps of the three serotypes, respectively (drawn with Snapgene); D: the molecular-weight size and the constitutional proportion of the capsid proteins VP1, VP2, and VP3 expressed by the three serotypes, which are detected with a VP1 antibody in Western blot.

FIG. 2 shows the comparison between the serotypes of the present invention (RC-C08, RC-C15, and RC-C18) and the existing serotypes (AAV2 WT and AAV2.7m8) in the viral yielding efficiency. FIGS. 2A and 2B are the statistical histogram plots for the viral yields of 5 serotypes obtained by packaging in adherent 293T cells and suspension 293 cells, respectively.

FIG. 3 shows the comparison of the in vitro biological activity (Transducing Unit, TU) between the three serotypes of the present invention and the two serotypes known in the prior art (as the control). The expression result statistics of the infections at multiple concentration gradients in the cultured 293T cells by five exogenous-gene (EGFP)-harboring serotypes AAV2 WT, AAV2.7m8, RC-C08, RC-C15, and RC-C18 are shown in A-E, respectively. FIG. 3F is the comprehensive analysis for the comparison of the VG/TU ratio of the five serotypes. *:p<0.05, **:p<0.01, ***:p<0.001, ****:p<0.0001, ns:no statistically significant difference.

FIG. 4 shows the comparison of the in vitro transduction activity between the serotype RC-C08 and the serotypes AAV2, AAV2.7m8, AAV-DJ. A-H show the histogram plots for the statistics of the percentage of GFP-positive cells and the mean fluorescence intensity in 4 different cells, respectively. *:p<0.05, **:p<0.01, ***:p<0.001, ****:p<0.0001, ns:no statistically significant difference.

FIG. 5 shows the comparison of the difference in the transduction efficiency between the serotype RC-C08 and the serotypes AAV2.7m8 & AAV-DJ after intravitreal (IVT) administration in the eyes of a mouse. A and D are the microscopy images for the intravital autofluorescence and the retinal flat mount. B-C and E-F are the statistical histogram plots for the fluorescence area and the fluorescence intensity in the two assays, respectively.

FIG. 6 shows the validation of the differences in the transduction activity in different cell lines between the three novel serotypes RC-C08, RC-C15, & RC-C15 and the two control serotypes AAV2 & AAV2.7m8. A-F show the histogram plots of the statistics of the percentage of GFP-positive cells and the mean fluorescence intensity in 3 different cells, respectively. *:p<0.05, **:p<0.01, ***:p<0.001, ****:p<0.0001, ns:no statistically significant difference.

FIG. 7 shows the comparison between the in vivo short-term (2 weeks) transduction activities of the serotype RC-C08 and the existing serotypes. A is autofluorescence from the back of the eyeball viewed under an intravital fluorescence microscope, and B-D are the statistical histogram plots of the total fluorescence area, the mean fluorescence intensity, and the total fluorescence intensity after infection with the three serotypes (the low dose groups). *:p<0.05, **:p<0.01, ***:p<0.001, ****:p<0.0001, ns:no statistically significant difference.

FIG. 8 shows the comparison of the in vivo tissue distribution and long-term activity validation between RC-C08 and AAV2.7m8. A and D are the microscopy images of the intravital autofluorescence (Week 5) and the fluorescence on the frozen sections (Week 6). B-C and E-F are the statistical histogram plots of the relative fluorescence area and the total fluorescence intensity in the two assays, respectively. *:p<0.05, **:p<0.01, ***:p<0.001, ****:p<0.0001, ns:no statistically significant difference.

FIG. 9 shows the comparison of the viral activity of tolerating the human neutralizing antibodies among RC-C08 and variants thereof. A-E are the plots and IC₅₀ values for suppresstion of the five serotypes AAV2 WT, AAV2.7m8, RC-C08, RC-C15, and RC-C18 by various concentrations of neutralizing antibodies, respectively, and FIG. 9F is comprehensive analysis for the comparison of the ability of escaping or tolerating the neutralizing antibody of the five serotypes.

FIG. 10 shows the comparison of the transduction efficiency of the two serotypes (RC-C08 and its variant RC-C15) in mouse ocular tissue. A and B are images taken under an intravital fluorescence microscope after infection with the two serotypes for a certain period of time, and C and D are the histogram plot for statistical analysis of the fluorescence area and the total fluorescence intensity at different points in time after infection with the two serotypes. *:p<0.05, **:p<0.01, ***:p<0.001, ****:p<0.0001, ns: No statistically significant difference.

### DETAILS OF INVENTION

As described in the present application, the present inventors have studied rAAV vectors as genetic drugs for treatment of ocular disorders for a long term, and have found surprisingly the following during this process: engineering in a capsid protein of the AAV2-based rAAV vector by introduction of amino acid residue modifications at least comprising the following: Q464V, A467P, D469N, I470M, R471A, D472V, S474G, Y500F, and S501A (the residue positions being numbered with reference to the amino acid sequence numbering for the VP1 protein of the native AAV2) will render the tropism and/or tissue specificity of the engineered rAAV vector to the retina tissue to be enhanced and/or the transduction efficiency thereof to be greatly increased. Consequently, in the first aspect, the invention provides a method of engineering an AAV2-based rAAV vector, wherein the rAAV vector is used in delivering an exogenous gene to local tissues of an individual (ocular tissues, for example, the retina) and comprises the sequence of an exogenous gene and the inverted terminal repeats (ITRs). The method comprises introduction of the following amino acid modifications into the capsid protein of the said rAAV vector: Q464V, A467P, D469N, I470M, R471A, D472V, S474G, Y500F, and S501A. In some other embodiments, the method of engineering is based on another respect of the present invention, namely on the basis of the 9 of the mutations as described above, further introducing the modifications Y444F and Y730F. In some other embodiments, the method of engineering is based on another respect of the present invention, namely on the basis of the 9 of the mutations as described above, further inserting an amino acid sequence LALGDVTRPA between the residue sites 587(N) and 588(R). In some preferred embodiments, the aforedescribed method of engineering by introducing amino acid mutations is realized by changing the nucleic acid sequence of the AAV cap gene to encode an amino acid sequence including the desired modification. In some preferred embodiments, changing the nucleic acid sequence of the gene is attained with one or more of molecular cloning means well known in the art.

In one aspect, the present invention provides a method of increasing the transduction efficiency of the AAV2-based rAAV vector after being delivered to ocular tissues (e.g., the retina). The method comprises engineering the rAAV vectors with the method of engineering in the first respect of the present invention. In one preferred embodiment, the present invention provides a method of increasing the transduction efficiency of the AAV2-based rAAV vector after being delivered to the retinal pigment epithelial cells. The method comprises engineering the rAAV vectors with the method of engineering in the first respect of the present invention. In another preferred embodiment, the present invention provides a method of increasing the transduction efficiency of the AAV2-based rAAV vector after being delivered to the retinal photoreceptor cells. The method comprises engineering the rAAV vectors with the method of engineering in the first respect of the present invention.

In one aspect, the present invention provides a method of increasing the infection proportion of the AAV2-based rAAV vector after being delivered to ocular tissues (e.g., the retina). The method comprises engineering the rAAV vectors with the method of engineering in the first respect of the present invention. In one preferred embodiment, the present invention provides a method of increasing the infection proportion of the AAV2-based rAAV vector after being delivered to the retinal pigment epithelial cells. The method comprises engineering the rAAV vectors with the method of engineering in the first respect of the present invention. In another preferred embodiment, the present invention provides a method of increasing the infection proportion of the AAV2-based rAAV vector after being delivered to the retinal photoreceptor cells. The method comprises engineering the rAAV vectors with the method of engineering in the first respect of the present invention.

In one aspect, the present invention provides a method of increasing the amount of expression of an exogenous gene from the AAV2-based rAAV vector after being delivered to ocular tissues (e.g., the retina). The method comprises engineering the rAAV vectors with the method of engineering in the first respect of the present invention. In one preferred embodiment, the present invention provides a method of increasing the amount of expression of an exogenous gene from the AAV2-based rAAV vector after being delivered to the retinal pigment epithelial cells. The method comprises engineering the rAAV vectors with the method of engineering in the first respect of the present invention. In another preferred embodiment, the present invention provides a method of increasing the amount of expression of an exogenous gene from the AAV2-based rAAV vector after being delivered to the retinal photoreceptor cells. The method comprises engineering the rAAV vectors with the method of engineering in the first respect of the present invention.

In one aspect, the present invention provides a method of reducing immunogenicity of the AAV2-based rAAV vector. The method comprises engineering the rAAV vectors with the method of engineering in the first respect of the present invention. In one aspect, the present invention provides a method of increasing tolerance or resistance of the AAV2-based rAAV vector to the pre-existing immunity (for example, but not limited to, neutralizing antibodies) in an individual. The method comprises engineering the rAAV vectors with the method of engineering in the first respect of the present invention.

In the first aspect, the invention provides an AAV2-based rAAV vector engineered with the method of the present invention, wherein the rAAV vector is used in delivering an exogenous gene to local tissues of an individual (e.g., ocular tissues, for example, the retina) and comprises the sequence of the exogenous gene and the inverted terminal repeats (ITRs). In some embodiments, the engineered rAAV vector has an engineered sequence for the capsid protein. In some embodiments, the capsid protein of the engineered rAAV vector has the following modifications in the amino acid sequence in comparison with that of the wild-type AAV2: Q464V, A467P, D469N, I470M, R471A, D472V, S474G, Y500F, and S501A. In some embodiments, the capsid protein of the engineered rAAV vector has the following modifications in the amino acid sequence in comparison with that of the wild-type AAV2: Y444F, Q464V, A467P, D469N, I470M, R471A, D472V, S474G, Y500F, S501A, and Y730F. In some embodiments, the capsid protein of the engineered rAAV vector has the following modifications in the amino acid sequence in comparison with that of the wild-type AAV2: Q464V, A467P, D469N, I470M, R471A, D472V, S474G, Y500F and S501A, and insertion of the amino acid sequence LALGDVTRPA between the residue sits 587(N) and 588(R). In some embodiments, the capsid protein VP1 of the engineered rAAV vector comprises the amino acid sequence set forth in any one of SEQ ID NOs: 1-3. In some embodiments, the engineered rAAV vector exhibits (i) the increased tropism to the retinal tissue; (ii) the enhanced specificity to the retinal tissue; (iii) the increased infection efficiency in cells of retinal tissue; (iv) the increased amount of expression of the exogenous gene in cells of the retinal tissue; and/or (v) the reduced immunogenicity. In some preferred embodiments, the immunogenicity of the engineered rAAV vector is reduced so that the vector is capable of tolerating or resisting to the higher level of pre-existing immunity against AAVs. In some preferred embodiments, the “higher” level refers to 5-fold higher, 10-fold higher, 20-fold higher, 30-fold higher, or 50-fold higher. In some embodiments, the pre-existing immunity is neutralizing antibodies. In some embodiments, the pre-existing immunity is T cell immunity.

In some embodiments, the present invention provides a cap gene encoding the amino acid sequence set forth in any one of SEQ ID NOs: 1-3. In a few further embodiments, the cap gene of the present invention has the nucleic acid sequence set forth in any one of SEQ ID NOs: 4-6.

In one aspect, the present invention provides a composition. The composition comprises any one or more of the rAAV vectors as described above in the present invention, and optionally one or more of pharmaceutical acceptable excipients. In some embodiments, the composition is a pharmaceutical composition. In some embodiments, the composition can be administered to an individual in a way of systemic, intravenous, intra-muscular, subcutaneously, oral, topical, topically contacting, intraperitoneal, or intralesional administration. In some embodiments, the composition can be administered to the individual in a way of eye drop instillation, intra-ocular, subconjunctival, intracameral, intravitreal, or subretinal injection.

In one aspect, the invention provides the use of any one or more of the rAAV vectors or the compositions as described above in preparation of a medication. In some embodiments, the medication is used to treat an ocular disorder. In some embodiments, the medication can be administered to an individual in a way of systemic, intravenous, intra-muscular, subcutaneously, oral, topical, topically contacting, intraperitoneal, or intralesional administration. In some embodiments, the medication can be administered to the individual in a way of eye drop instillation, intra-ocular, subconjunctival, intracameral, intravitreal, or subretinal injection.

In one aspect, the invention provides a method for treating a disease. The method comprises administering the rAAV vectors engineered through the method of the present invention or the rAAV vectors of the present invention to an individual with the disease. In some preferred embodiments, the disease is an ocular disorder. In some more preferred embodiments, the disease is a disorder caused by retinopathy. In some more preferred embodiments, the disease is IRD.

### I. Prior Art

The Chinese Patent Publication No. CN107012171B is related to a variant designated AAV2.7m8, which is engineered from a known AAV2 serotype and in which the amino acid at the position 588 of the AAV2 capsid protein VP1 is replaced with a peptide of 11 amino acids targeting the retinal tissue, resulting in alteration of the conventional capability of AAV2 virus capsid binding to the HSPG receptor. The patent is hereby incorporated herein by reference in its entirety, and the sequence of the AAV2.7m8 capsid protein is particularly listed herein set forth in SEQ ID NO: 7. AAV2.7m8 represents an attempt in the prior art for delivering rAAV vectors to the eye, especially to the retina, in order to express efficiently an exogenous gene. The variant is herein cited only for the research purpose, and unless otherwise stated, will serve together with the wild-type AAV2 as the prior-art control, to verify if the methods and the vectors of the present invention have been significantly improved relative to the prior art. In addition to this section, citation of the variant throughout the disclosure will also be indicated by the annotations such as “AAV2.7m8”, “CN107012171B”, etc.

### II. Definitions

The term “about” used in combination of a numerical value is intended to encompass the numerical values in a range from a lower limit less than the specified numerical value by 5% to an upper limit greater than the specified numerical value by 5%.

As used in herein, the terms “comprising” or “including” are intended to include a stated element, integer or step, but not exclude any other element, integer or step.

The term “encoding” refers to the inherent property of specific sequences of nucleotides in a nucleic acid, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene, cDNA, or RNA encodes a protein if transcription and translation of mRNA corresponding to that gene produces the protein in a cell or other biological system. Both the coding strand (the nucleotide sequence of which is identical to the mRNA sequence and is usually provided in the sequence listing) and the non-coding strand (used as the template for transcription of a gene or cDNA) can be referred to as encoding the protein or other products of that gene or cDNA.

The terms “protein” and “polypeptide” are used interchangeably herein and refer to the sequence of a polymer comprising amino acid residues. The single-letter and 3-letter codes for amino acids as defined in conformity with the IUPAC-IUB Joint Commission on Biochemical Nomenclature (JCBN) is used throughout this disclosure, unless otherwise stated. A single-letter X refers to any one of the twenty amino acids. It should also be understood that a polypeptide can be encoded by one more nucleotide sequences due to the degeneracy of the genetic codes. Mutation in the amino acid sequence can be designated as follows: single-letter code for a parental amino acid, followed by the number for the position, then followed by the single-letter code for a variant amino acid. For example, mutation of glutamine (Q) at Position 464 into valine (V) is represented as “Q464V”.

“Homology” refers to the identity percentage between portions of two polynucleotides or two polypeptides. When referring to amino acids or fragments thereof, the term “substantial homology” indicates that when optimally aligned with appropriate nucleotide insertions or deletions with another nucleic acid (or its complementary strand), there is nucleotide sequence identity in about 90% to 100% of the aligned sequences. When referring to polypeptides or fragments thereof, the term “substantial homology” indicates that when optimally aligned with appropriate gaps, insertions or deletions with another polypeptide, there is nucleotide sequence identity in about 90% to 100% of the aligned sequences. The term “highly conserved” means at least 80% identity, preferably at least 90% identity, and more preferably, over 97% identity. In some situations, highly conserved can refer to 100% identity. Identity is readily determined by those of skill in the art by for example using algorithms and computer programs known by those of skill in the art.

As described in herein, any of multiple sequence alignment programs publically or commercially available (e.g., “Clustal W” accessible through web servers on the internet) is used to perform alignment between the nucleic acid or polypeptide sequences. Alternatively, the application Vector NTI can also be used. There are also a number of algorithms known in the art which may be useful for measuring nucleotide sequence identity, including those inclined in the programs described above. As another example, BLASTN can be used to compare polynucleotide sequences and it provides alignments and the sequence identity percentage of the best-overlapping region between the query and search sequences. Similar programs may be useful for comparing amino acid sequences, for example, “Clustal X” program, BLASTP. In general, any one of these programs is used at default settings, although those of skill in the art can alter these settings as needed. Alternatively, those of skill in the art may use another algorithm or computer program which provides at least the level of identity or alignment as that provided by the referenced algorithms and programs. Alignment can be used to recognize the corresponding amino acids between two proteins or peptides. The “corresponding amino acids” are amino acids in the sequence of a protein or peptide which are aligned with those in the sequence of another protein or peptide. The corresponding amino acids may be the same or different. The corresponding amino acids which are different amino acids can be designated as variant amino acids.

Alternatively, for nucleic acids, homology can be determined via the following process: hybridizing polynucleotides under conditions that allow formation of stable duplexes between homologous regions, followed by digestion with single-stranded-specific nuclease(s) and size determination of the digested fragments. DNA sequences that are substantially homologous can be identified in a Southern hybridization experiment under stringent conditions (for example, as defined for that particular system). Defining appropriate hybridization conditions is within the skill of the art.

The nucleic acid and protein sequences described herein can be used as a “query sequence” to perform a search against public databases, for example, to identify other family members or related sequences.

The term “isolated” as used herein means artificially obtained or generated. The term “isolated” as used herein about nucleic acids refers typically to: (i) amplified in vitro by, for example, polymerase chain reaction (PCR); (ii) recombinantly generated by cloning; (iii) purified through cleavage and gel fractionation; or (iv) synthesized by, for example, chemical synthesis. An isolated nucleic acid is one which can be easily manipulated by recombinant DNA techniques well known in the art. Thus, a nucleotide sequence contained in a vector in which 5′ and 3′ restriction sites are known or for which primer sequences in polymerase chain reaction (PCR) have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. However, such a nucleic acid is also isolated, as that term is used herein, because it may be easily manipulated by standard techniques known to those of ordinary skill in the art. The term “isolated” as used herein about proteins or peptides refers typically to a protein or peptide artificially obtained or generated (e.g., by chemical synthesis, by recombinant DNA technology, etc.). In some embodiments, proteins and nucleic acids of the invention are isolated.

“Host cells” refers to any cell that accommodates or is capable of accommodating a substance of interest. Typically a host cell is a mammalian cell. A host cell can be used as the recipient for AAV helper constructs, AAV small gene plasmids, accessory function vectors or other relevant transferred DNA produced together with recombinant AAVs. The term includes the progeny of the original cell which has been transfected. Thus, a “host cell” used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

In some aspects, the invention provides a transfected host cell. The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary individual cell and its progeny. “Infection” is a particular form of “transfection” or “transformation” or “transduction” in which exogenous nucleic acid is transferred or introduced into the host cell with the help of pathogenic agents (e.g. viruses). An “infected” cell is one which has been transfected, transformed or transduced with pathogenic agents, for example, viruses (e.g. lentiviruses).

The term “transducing unit (TU)” used with reference to viral valence is intended to refer to the number of infectious particles of the recombinant AAV vector determined in a functional assay, which infectious particles lead to production of functional transgenic products.

The term “vector genome (vg)” can refer to one or more of polynucleotides comprising a set of polynucleotide sequences of the vector (e.g. a viral vector). The vector genome can be encapsidated into viral particles. Depending upon a particular viral vector, a vector genome can comprise single stranded DNAs, double stranded DNAs or single-stranded RNAs or double-stranded RNAs. The vector genome can comprise endogenous sequences associated with the particular viral vector and/or any heterologous sequence inserted into the particular viral vector by recombination technologies. Fr example, the recombinant AAV vector genome can comprise at least one ITR sequence located at the flanking side to a promoters, a spacing fragment, an exogenous gene and a polyadenylation sequence. A whole vector genome can comprise the whole set of polynucleotide sequences of the vector. In some embodiments, the infection efficacy of viral vectors may be measured with VG/TU. Methods suitable for measurement are known in the art.

The term “multiplicities of infection (MOI)” mean the ratio of the quantity of virus to the number of cells when infection occurs. Although the ratio varies with the types of viruses and cells, even with the influence of factors such as cultivation conditions, those of skill in the art may determine easily the MOI of a virus for a cell type with a well-known and conventional method, to attain the experimental purpose.

The term “cells line” as used herein refers to a cell population capable of in vitro continuous or extended growth and division. In general, a cells line is derived from the clone population of a single progenitor cell. It is further known that, during the storage or translocation of that clone population, karyotypes may undergo spontaneous or induced changes. Therefore, cells derived from the mentioned cell line may be not completely identical to the progenitor cell or the culture; and the mentioned cell line includes these variants.

Cells may further be transfected with a vector providing helper functions for AAVs (e.g., a helper vector). The vector providing helper functions can provide adenovirus helper functions, including, for example, E1a, E1b, E2a, and E4ORF6. The sequences of adenovirus genes providing these functions can be obtained from any of the known adenovirus serotypes, for example, the serotypes 2, 3, 4, 7, 12 and 40, and further including any of the currently identified human types known to the art. Accordingly, in some embodiments, the method comprises transfection of cells with a vector expressing one or more genes required for replication of AAVs, transcription of AAV genes and/or packaging of AAVs.

As used herein, when referring to genetic engineering or molecular clone technologies, the term “vector” refers to any nucleic acid molecule and/or nucleic acid/protein complex, such as plasmid, phage, transposon, cosmid, chromosome, artificial chromosome, virus, virion, etc., which can transfer gene sequences into cells of interest and preferably is capable of replication when its interacting with the proper control elements or bioactive molecules within host cells. Thus, the term includes vectors for cloning and expression, as well as viral vectors. In some preferred embodiments, the gene sequence to be transferred in the vector (typically designated as the exogenous gene sequence) is located under transcriptional control of a promoter, transcribed in the host cell at appropriate time points and circumstances, and eventually expressed to yield a protein. The term “operably located”, “under control” or “under transcriptional control” refers to location of a promoter in a proper position and orientation relative to a nucleic acid to control initiation of RNA polymerases and expression of genes.

The term “operably linked” means that the components designated are in a relationship permitting them to function in their intended manner.

The terms “regulatory sequence” or “expression control sequence” refers to a nucleic acid sequence which induces, suppresses or otherwise controls transcription of an encoding nucleic acid sequence operably linked thereto into proteins. The regulatory sequence may be for example a starting sequence, an enhancer sequence, an intronic sequence, a promoter sequence and the like.

The term “expression vector or construct” refer to any type of genetic constructs comprising a nucleic acid of which part or all of the encoding sequence is able to be transcribed. In some embodiments, expression includes transcription of nucleic acids, for example, producing biologically active polypeptide products or inhibitory RNAs (e.g., shRNAs, miRNAs, and miRNA suppressors) through the transcribed genes.

### III. rAAV Vectors

As used in herein, the term “adeno-associated virus (AAV)” takes its name from its presence in adenovirus preparations. AAV is a member of Parvoviridae and comprises various serotypes, with a single-stranded DNA genome. AAV is a replication-defective non-enveloped virus, replication of which is dependent upon the presence of the second virus, such as adenoviruses, HPVs, or herpes viruses, or auxiliary functional proteins provided by auxiliary factors. Currently, it has not been found currently for AAVs to cause a disease in human being, and therefore AAVs induce merely the very slight immunological responses in human bodies. AAV can infect both dividing and non-dividing cells. The prototypic AAV vector based on the serotype 2 is avirulent and stable gene transfer provides a proof-of-concept. However, the efficiency of gene transfer shown in many major target tissues is insufficient. In some aspects, the present invention try to overcome this disadvantage by providing a novel AAV with unique tissue-targeting competence, for use in gene therapy and research applications.

Although as described above, it has not been found currently for AAVs to cause a disease in human being, and therefore AAVs induce merely the very slight immunological responses in human bodies. However, an individual who had been infected with an AAV or underwent a rAAV gene therapy is still likely to retain an immune response specific to the AAV in the body, namely, pre-existing immunity. The pre-existing immunity may be either B cell immunity (neutralizing antibodies) or T cell immunity. And the pre-existing immunity may be cross-reactive, namely the pre-existing immunity raised by the first AAV serotype is also likely to be against the posterior second AAV. The presence of pre-existing immunity is still an obvious hindrance to a viral vector's acting as a tool in gene therapy.

The AAV virus isolated earliest is the AAV serotype 2 (AAV2). The genome of the AAV2 is about 4.7 kb in length and there are inverted terminal repeats (ITRs) of 145 bps in length in palindrome-hairpin structure at the two ends of the genome In the genome, there are two large Open Read Frames (ORFs), encoding for the Rep and Cap genes, respectively.

The ITRs are cis-acting elements of the AAV vector genome, playing an important role in integration, rescue, replication and genome packaging of AAV virus. In the ITR sequences are contained Rep binding sites (RBS), which can be bond to and recognized by Rep proteins, and terminal resolution sites (TRSs), at which nicks are made. The ITR sequences can also form a unique “T letter”-shaped secondary structure which plays an instrumental role in the life cycle of AAV virus. In embodiments of the present invention, ITR sequences of any serotype known in the art may be utilized. In some preferred embodiments, the present invention utilizes ITR sequences from the AAV serotype 2.

The remainder of the AAV2 genome can be divided into two functional regions, i.e., Rep gene region and Cap gene region.

The Rep gene region encodes four proteins: Rep78, Rep68, Rep52, and Rep40. Rep proteins are essential for replication, integration, rescue, and packaging of AAV virus. Among them, Rep78 and Rep68 bind specifically to the terminal resolution sites (TRSs) and the GAGY repeat motif in the ITRs, initiating the replication process in which the AAV genome turns from single-stranded to double-stranded. The TRSs and GAGC repeat motif and/or GAGY repeat motif in the ITRs are the core for replication of the AAV genome. Therefore, although sequences of the ITRs in various serotypes of AAV viruses are varied, they all can form the hairpin structure and enable existence of the Rep binding sites. The p19 promoter is at Position 19 of the AAV2 genomic map, initiating expression of Rep52 and Rep40, respectively. Rep52 and Rep40 have ATP-dependent DNA helicase activities, but no DNA-binding functions.

The Cap gene encodes for the AAV virus capsid proteins VP1, VP2 and VP3. Among these proteins, VP3 has the lowest molecular-weight and the highest amount. In mature AAV particles, the ratio for VP1, VP2, and VP3 is about 1:1:10. VP1 is essential for forming infectious AAVs; VP2 assists in VP3 entering into the cellular nucleus; VP3 is the major protein constituting AAV particles. An exemplary sequence of AAV2 VP1 can be referred to NCBI Reference Sequence YP_680426, i.e., SEQ ID NO: 8 of the application. The wild-type sequences of VP1 protein from other serotypes or the wild-type sequences of VP2 and VP3 proteins from any serotype can be found readily in a manner known to those of skill in the art from bioinformatic databases (e.g. NCBI Genbank and so forth).

As used in herein, the terms “recombinant AAV (rAAV) vector” refers to an efficient tool for transferring exogenous genes, which is obtained by engineering of the wild-type AAV virus on the basis of understanding the life cycle of the AAV virus and the relevant mechanism thereof in molecular biology, namely rAAV vector. In the rAAV vector genome are only contained ITR sequences of the AAV virus and an expression cassette harboring exogenous genes to be transferred. The Rep and Cap proteins required for AAV virus packaging are not incorporated into the rAAV vector genome, but provided by other exogenous plasmids, hence reducing the hazard potentially imposed by packaging the Rep and Cap genes into the rAAV vector.

The present invention provides a method for improving rAAV vectors, characterized in that, on the basis of the wild-type AAV2 sequence, modification of amino acid residues is conducted (for example, but not limited to, substitution, deletion and/or addition), resulting in the changed affinity of the modified AAV2 vector toward one or more of the receptors on the target cell's surface. Therefore in a preferred embodiment, the method is a method for engineering an AAV2 capsid protein. In a more preferred embodiment, the method is to engineer the AAV2 capsid protein VP1 to have the following amino acid mutations: Q464V, A467P, D469N, I470M, R471A, D472V, S474G, Y500F, and S501A. In another more preferred embodiment, the method is to engineer the AAV2 capsid protein VP1 to have the following amino acid mutations: Q464V, A467P, D469N, I470M, R471A, D472V, S474G, Y444F, Y500F, S501A, and Y730F to have the following amino acid mutations: Q464V, A467P, D469N, I470M, R471A, D472V, S474G, Y500F, and S501A, simultaneously with the amino acid sequence LALGDVTRPA inserted between 587N and 588R.

The AAV capsid proteins of the present invention include any protein having the amino acid sequence set forth in any one of SEQ ID NOs: 1-3 and any protein substantially homologous thereto. In some embodiments, the invention provides an isolated capsid protein which is substantially homologous to a protein having the sequence set forth in any one of SEQ ID NOs: 1-3, but does not has the amino acid sequence set forth in SEQ ID NO: 8 (i.e., of the capsid protein VP1 of the wild-type AAV2).

Isolated nucleic acid molecules encoding for the AAV capsid protein of the present invention include any nucleic acid molecule having the sequence set forth in any one of SEQ ID NOs: 4-6 and any nucleic acid molecule having the sequence substantially homologous thereto. In some preferred embodiments, the nucleic acid molecule is the cap gene. In some embodiments, the invention provides an isolated nucleic acid which is substantially homologous to a nucleic acid molecule having the sequence set forth in any one of SEQ ID NOs: 4-6, but does not encode for the protein having the amino acid sequence set forth in SEQ ID NO: 8 (i.e., the capsid protein VP1 of the wild-type AAV2).

Fragments of the isolated nucleic acid molecule encoding for sequences of the AAV capsid may be useful for construction of nucleic acids encoding for the sequences of the intended capsid. The fragment can be in any appropriate length. In some embodiments, fragments (i.e., portions) of the isolated nucleic acid encoding for sequences of the AAV capsid may be useful for construction of nucleic acids encoding the sequences of the intended capsid proteins. The fragment can be in any appropriate length (e.g., at least 6, at least 9, at least 18, at least 36, at least 72, at least 144, at least 288, at least 576, at least 1152, at least 1728 or more nucleotides in length). By incorporating fragments of a nucleic acid sequence encoding for the region of variant amino acids into the nucleic acid sequence of a known AAV serotype, a recombinant cap sequence can be constructed to encode for a variant capsid protein having the desired amino acid modifications. The said fragment can be incorporated by any suitable method, including by use of, for example, site-directed mutagenesis.

In some embodiments, the capsid proteins are the structural proteins encoded by the AAV cap gene. In some embodiments, AAV comprises three capsid proteins: the virosome proteins 1 to 3 (VP1, VP2, and VP3), all of which can be expressed from the single cap gene. Consequently, in some embodiments, VP1, VP2, and VP3 proteins share a common core sequence. In some embodiments, molecular weights for VP1, VP2, and VP3 are about 87 kDa, about 72 kDa and about 62 kDa, respectively. In some embodiments, after translation, the capsid proteins form a spherical 60-mer proteinaceous shell surrounding the viral genome. In some embodiments, the functions of the capsid proteins are to protect the viral genome, delivery the genome and interact with the host. In some aspects, the capsid proteins deliver the viral genome to the host in a tissue-specific manner. In some embodiments, the VP1 capsid protein is key to the tissue tropism of the packaged rAAV vector. In some embodiments, the tissue tropism of the AAV is enhanced or altered by mutations occurring in the capsid proteins.

In some aspects, the present invention describe variants of wild-type AAV serotypes. In some embodiments, the variant has an altered tissue tropism. In some embodiments, AAV variants described herein comprise amino acid changes in the cap gene, for example, but not limited to, substitutions, deletions (namely, deleting), additions (namely, insertions). In some embodiments, amino acid changes occur only in VP1 capsid protein; in some embodiments, only in VP1 and VP2 capsid proteins; in some embodiments, only in VP1 and VP3 capsid proteins; in some embodiments, in all of the 3 capsid proteins.

In some embodiments, the AAV variants of the present invention may be useful for delivering gene therapy to ocular tissues. Accordingly, in some embodiments, the AAV variants described herein may be useful for treating ocular disorders. An ocular disorder may be of a genetic origin, either inherited or acquired through a somatic mutation. In some preferred embodiments, the rAAV vectors of the present invention may be useful for delivering gene therapy to human retinal tissue cells (e.g., RPE and/or photoreceptor cells). Hence, the rAAV vectors of the present invention may be useful for treating retinopathy.

In some aspects, the present invention provides an isolated rAAV. Methods for obtaining rAAV are well-known in the art. There are packaging systems which are relatively mature relative to rAAV vectors in the prior art, which facilitates manufacturing rAAV vectors in scale.

It is known in the art that many methods find use for packaging and producing rAAV vectors. At present, the packaging systems for rAAV vectors commonly used include mainly the three-plasmid co-transfection system, the system having adenovirus as the helper virus, the packaging system having herpes simplex virus (Herpes simplex virus type 1, HSV1) as the helper virus and the baculovirus-based packaging system. Each of the packaging systems has its own characteristics and those of skill in the art can make a suitable choice as needed. The rAAV production cultures for producing rAAV virus particles are all need: 1) suitable host cells, include for example cells lines originating from human being, such as, HEK-293 cells, or cells lines originating from insects (in case for baculovirus-based manufacturing systems); 2) functionality from suitable helper viruses, provide by the wild-type or mutant adenoviruses (such as a temperature susceptible adenovirus), herpes viruses, baculoviruses, or plasmid constructs providing auxiliary functions; 3) AAV's rep and cap genes and gene products; 4) exogenous genes, flanked by at least one AAV ITR sequence and preferably driven under operably linked promoters; and 5) suitable cultivation system, to support manufacturing of rAAV. Suitable media known in the art may be useful for producing rAAV vectors.

An “exogenous gene” is meant to indicate a fragment derived from a nucleic acid sequence that is distinct in genotype from the other genes which are compared to the nucleic acid sequence or which have been introduced or integrated into nucleic acids/vectors/host cells and the like. For example, a polynucleotide introduced into different cell types by genetic engineering techniques is an exogenous gene, which encodes and expresses an exogenous polypeptide. Similarly, a cellular sequence (e.g. a gene or a portion thereof) incorporated into a viral vector is exogenous with respect to the vector. It can be understood by those of skill in the art that, in most cases, introduction of an exogenous gene is on the basis of the substantially lacking the gene and/or the functionality thereof at the introduction site, thus the gene being introduced as desired, and leads to the substantial change (e.g., a significant increase) in the number and/or functionality of the gene. In this disclosure, unless otherwise stated, the terms “exogenous gene” and “gene of interest (GOI)” as understood in the context indicate the same meaning and can be used interchangeably. In one preferred embodiment, the exogenous gene of the present invention is a green fluorescent protein gene EGFP. The gene is a reporter gene used commonly in the art and will produces a green fluorescent protein upon being expressed in a eukaryotic cell. When elicited under an appropriate wavelength, the protein emits green fluorescence, thus rendering an experimenter to acquire the expression area and/or amount of the protein in a detectable manner, so as to directly evaluate, for example, transduction efficiency, infection efficiency, expression efficiency for a population of cells, transduction result in a single cell, location of the protein expressed, and so forth. Methods for detecting the green fluorescence protein are well known in the art, and the required reagents/instrumentation are readily available in the field (e.g., commercially acquired).

The components to be cultured in a host cell to package a rAAV vector into an AAV capsid may be provided in trans to the host cell. Alternatively, any one or more of the required components (e.g., the recombinant AAV vector genome, Rep sequences, Cap sequences, and/or helper functions) may be provided by a stable host cell which has been engineered to contain one or more of the required components using methods known to those of skill in the art. For example, a stable host cell may be generated which is derived from 293 cells (which contain E1 helper functions under the control of a constitutive promoter), but which contains the rep and/or cap proteins under the control of inducible promoters. Still other stable host cells may be generated by those of skill in the art.

The three-plasmid transfection packaging system is the most extensively used rAAV vectors packaging system due to no need of helper viruses and high safety, and also is currently the world-wide mainstream manufacturing system. It is somewhat insufficient that lack of efficient and large-scale transfection methods limits application of the three-plasmid transfection system in large-scale preparation of rAAV vectors.

### IV. Methods of Treatment

The term “treatment” refers to clinical intervention in an attempt to alter the natural course of a disorder in an individual being treated. Desirable effects of treatment include, but are not limited to, preventing occurrence or recurrence of disease, alleviation of symptoms, diminishing in any direct or indirect pathological consequences of the disease, decreasing the rate of disease progression, amelioration or palliation of the disease state, and remission or improved prognosis. When used herein, “prophylaxis” includes preventing or suppressing occurrence or development of a disorder or symptoms of a particular disorder.

rAAVs can be delivered in a composition to a subject according to any appropriate method known in the art. The rAAV, preferably suspended in a physiologically compatible carrier (e.g., in a composition), may be administered to a subject, for example, a host animal, such as a human, mouse, rat, cat, dog, sheep, rabbit, horse, cow, goat, pig, guinea pig, hamster, chicken, turkey, or a non-human primate (e.g., Macaque).

In view of the purpose of rAAV applications, suitable pharmaceutically acceptable excipients may be readily selected by those of skill in the art. For example, a suitable excipient includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of excipients is not a limitation of the present invention.

Optionally, the composition of the present invention may further contain other conventional pharmaceutical ingredients, such as preservatives, or chemical stabilizers diminishing Suitable exemplary preservatives include chlorobutanol, potassium sorbate, sorbic acid, suphur dioxide, propyl gallate, parabens, ethyl vanillin, glycerine, phenol and p-chlorophenol. Suitable chemical stabilizers include gelatin and albumin.

Formulation of pharmaceutically-acceptable excipients is well-known to those of skill in the art, as is the development of suitable dosing and treatment regimens for using the particular compositions described herein in a variety of treatment regimens.

The rAAV(s) are administered in sufficient amounts to transfect the cells of a desired tissue and to provide sufficient levels of gene transfer and expression without undue adverse effects. Conventional and pharmaceutical acceptable routes of administration comprise, but are not limited to.

Dose of the rAAV virosome required to attain a particular “effect of treatment” (e.g., genome copies/kilogram of body weight (GC/kg)) will be varied with a plurality of factors, comprising, but not limited to: routes of administration for the rAAV virosome, expression levels of genes or RNAs required to attain the effect of treatment, particular diseases or disorders being treated, and stability of genes or RNA products. Those of skill in the art may readily determine the dose range of a rAAV virosome for treating a patient with a particular disease or disorder on the basis of the factors abovementioned, as well as other factors well known in the art.

An effective amount of the rAAV is an amount sufficient to target the infected animals and the intended tissues. In some embodiments, an effective amount of the rAAV is an amount sufficient to generate stable somatic transgenic animal models. The effective amount will be largely dependent on the factors, such as the species, years of age, weight, healthy of the subject and tissues to be targeted, and thus may be varied among animals and tissues. For example, an effective amount of the rAAV is about 1 ml to about 100 ml of a solution typically comprising about 10⁹ to 10¹⁶ genome copies. In some embodiments, the rAAV is administered at a dose of 10¹⁰, 10¹¹, 10¹², 10¹³, 10¹⁴, or 10¹⁵ genome copies/subject. In some embodiments, the rAAV is administered at a dose of 10¹⁰, 10¹¹, 10¹², 10¹³, 10¹⁴, or 10¹⁴ genome copies/kg. In some situations, a dose of about 10¹¹ to 10¹² rAAV genome copies is suitable.

In general, these formulations may comprise at least about 0.1% or more of the active compound, although the percentage of the active ingredient(s) may, of course, be varied and may conveniently be between about 1% or 2% and about 70% or 80% or more of the weight or volume of the total formulation. Naturally, the amount of an active compound in each therapeutically-useful composition may be prepared in such a way that a suitable dose will be obtained in any given unit-dose of the compound. Factors such as solubility, bioavailability, biological half-life, route of administration, product shelf life, as well as other pharmacological considerations will be contemplated by one skilled in the art of preparing such pharmaceutical formulations, and thus, a variety of dosages and treatment regimens may be intended.

In some embodiments, a rAAV-based therapeutic construct is intended to be delivered intraocularly in an appropriately formulated pharmaceutical composition as disclosed herein. In some embodiments, a preferred mode of administration is through intravitreal injection.

Pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases, the said form is sterile; and fluidity attains the degree of easy injection. It must be stable under the conditions of manufacture and storage and must be preserved against the contaminating action of microorganisms, such as bacteria and fungi. The carrier can be a solvent or dispersion medium containing, for example, water, ethanol, polyols (e.g., glycerol, propylene glycol, liquid polyethylene glycol, etc.), suitable mixtures thereof and/or vegetable oils. Proper fluidity may be maintained, for example, by use of a coating, such as lecithin, by maintenance of the required particle size in the case of dispersion and by use of surfactants. Prevention of the action of microorganisms may be attained with various antibacterial and antifungal agents, e.g. methyl p-hydroxybenzoates, chlorobutanol, phenol, sorbic acid, thiomerasol, and the like. In many cases, it will be preferable to include isotonic agents, for example, sugars or sodium chloride. Prolonged absorption of the injectable compositions can be attained by using an agent which delays absorption in the composition, for example, aluminum monostearate and gelatin.

For administration of an injectable aqueous solution, for example, the solution may be suitably buffered, if necessary, and the liquid diluent is first rendered isotonic with sufficient saline or glucose. These particular aqueous solutions are especially suitable for intravenous, intramuscular, subcutaneous, and intraperitoneal administration. In this connection, a sterile aqueous medium that can be used will be known to those of skill in the art.

A sterile injection solution is prepared as follows: incorporating the required amount of the active rAAV into an appropriate solvent together with a variety of other ingredients enumerated herein, as required, followed by filtered sterilization. Generally, dispersions are prepared by incorporating the various sterilized active ingredients into a sterile carrier which contains the basic dispersion medium and/or the other required ingredients from those enumerated above. In the case of sterile powders for the preparation of sterile injectable solutions, the preferred methods of preparation are vacuum drying and freeze-drying techniques that yield a powder of the active ingredients plus any additional desired ingredients from a previously sterile-filtered solution thereof.

The rAAV compositions disclosed herein may also be formulated as a neutral or salt form. Pharmaceutically-acceptable salts include the acid addition salts (formed with the free amino groups of the protein) which are formed with inorganic acids such as hydrochloric or phosphoric acid, or organic acids such as acetic, oxalic, tartaric, mandelic acid, and the like. Salts formed with the free carboxyl groups can also be derived from inorganic bases for example, sodium, potassium, ammonium, calcium, or ferric hydroxides, and from organic bases such as isopropylamine, trimethylamine, histidine, procaine and the like. Upon formulation, solutions will be administered in a manner compatible with the dosage formulation and in therapeutically effective amounts. The said formulations are easily administered in a variety of dosage forms such as injectable solutions, drug-release capsules, and the like.

For introduction of pharmaceutically acceptable formulations of the nucleic acids or rAAV constructs disclosed herein, the formulations may be preferably, for example, liposomes.

Alternatively, nanocapsule formulations of the rAAV may be used. Nanocapsules can generally entrap substances in a stable and reproducible way. To avoid side effects caused by intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### V. Compositions and Kits

In the preferred embodiment, rAAVs and pharmaceutically acceptable excipients described herein exist in the form of compositions. In the preferred embodiment, the compositions are pharmaceutical compositions. In the preferred embodiment, the pharmaceutical compositions comprise

The compositions or pharmaceutical compositions can be assembled into medications or diagnostic or investigational kits to facilitate their use in treatment, diagnosis or study. The kits may include one or more containers holding the components of the invention and instructions for use. Specifically, such the kits may comprise one or more of the agents described herein and the instructions describing the intended applications and appropriate uses of these agents. In certain embodiments, agents in the kit may be in a pharmaceutical formulation and dosage suitable for a particular application and for a method of administration of the agents. Kits for research purpose may comprise appropriate concentrations or amounts of constituents to be useful in performing various experiments.

In some other aspects of the invention, provided is a kit which comprises a container holding any of the aforedescribed recombinant AAV vectors engineered by the method according to the present invention, or the recombinant AAV vectors of the present invention, or the pharmaceutical compositions of the present invention. In some embodiments, the container in the kit is an injector.

### VI. Pharmaceutical Use

In some other aspects of the invention, provided is the use of the recombinant AAV vector, the pharmaceutical composition, and/or the kit of the present invention as specified above, in preparation of a medication for treating diseases. In a few preferable technical solutions, the disease is an ocular disorder, for example retinopathies. In some more preferred technical solutions, the disease is IRD. In some embodiments, the medication is prepared as appropriate for systemic, intravenous, intra-muscular, subcutaneously, oral, topical, topically contacting, intraperitoneal, or intralesional administration. In some preferred embodiments, the medication is prepared in a manner suitable for administration by eye drop instillation, intra-ocular, conjunctival, intracameral, intravitreal, or subretinal injection.

The technical solutions of the present invention are described clearly and completely below in conjunction with the accompanying drawings and Examples, and only intended to help those of skill in the art understand the invention, but not to constitute any limitation on the embodiments of the invention. It is apparent that the described Examples are only a part of, not all of, the examples of the present invention. Based on the Examples of the present invention, all of the other examples obtained by a person with ordinary knowledge in the art without inventive efforts are well within the scope of protection of the invention. Although the detailed annotations have been made, experimental approaches for which particular conditions are not documented in details in the Examples are conventional techniques well known in the art, or are operated according to the instructions provided by the producers of reagents/kits.

Unless otherwise stated, “wild-type AAV2”, “wild-type AAV2-based rAAV vector”, “AAV2”, “AAV2 WT” in the following Examples indicate the same meaning and can be used interchangeably, indicative of a rAAV vector generated recombinantly on the basis of the natural AAV2 serotypes, which has capsid proteins having the same amino acid sequences as those of the wild-type AAV2 and not engineered with any of artificial methods.

### Example 1: Construction of Plasmids for the Three Serotypes RC-C08, RC-C15, RC-C18 and Detection of Viral Packaging and Molecular-weight of the Capsid

### Experimental Process:

1) Sequence Design and Acquisition of Cap gene: The present inventors have studied long-term the AAV2 capsid protein VP1 sequence and spatial structure and discover that the affinities of the capsid protein for different receptors can be adjusted by performing the targeted and rational designation on particular regions of the capsid protein and altering the motifs binding to different receptors. Furthermore, according to the search and prior experiments by the present inventors, it has been found that receptors, which interacts with AAV and aids in entrance of the latter into cells, exhibit such expression profiles in ocular tissues, especially in the retinal tissue, that are different from those in the other tissues. Hence, the aforesaid adjustment on the affinities of the capsid protein for its receptors may help to optimize the tissue tropism and/or the specificity of the viral particles in ocular tissues, especially in the retinal tissue, and this would further optimize the transduction efficiency and expressing efficiency for the exogenous genes. Therefore, on the basis of the abovementioned discovery, the present inventor have designed the novel serotypes: RC-C08, RC-C15, and RC-C18.

The sequence of the capsid protein VP1 of RC-C08 serotype is shown in SEQ ID NO: 1, comprising nine mutations as follows: Q464V, A467P, D469N, I470M, R471A, D472V, S474G, Y500F, and S501A, and the sequence of the corresponding cap gene is shown in SEQ ID NO: 4. Two more mutations Y444F and Y730F are added into the capsid protein VP1 of RC-C15 serotype on the basis of RC-C08; and for the RC-C18 serotype, the amino acid sequence LALGDVTRPA is further inserted into the capsid protein VP1 between 587N and 588R, on the basis of RC-C08. The sequences of the capsid proteins VP1 of RC-C15/C18 serotypes are shown in SEQ ID NO: 2 and 3, respectively, and the sequences of the corresponding cap genes are shown in SEQ ID NO: 5 and 6, respectively.

To obtain the viruses as designed above, the Cap genes of the serotypes RC-C08/C15/C18 were synthesized respectively by the trusted Shanghai Langjing Biotechnology Co. Ltd., with the cloning sequences added upstream and downstream thereof, and were T-A cloned into pUC57 vector to serve as a template for subsequent amplification. The resulting vectors are designated pUC-RC-C08/C15/C18 serotype Cap, respectively.

Fragments of the genes of interest were amplified with the PCR reaction system as shown in Table 1 below, for the purpose of subsequent cloning steps:

TABLE 1

| Constituents (purchased | Reaction system | | Final | |
|---|---|---|---|---|
| from Takara company) | of 50 μl | | concentration | |
| 5 × PrimeSTAR Buffer (Mg²⁺ Plus) | 10 | μL | 1X | |
| 10 μM Forward primer | 2.5 | μL | 0.5 | μM |
| 10 μM Reverse primer | 2.5 | μL | 0.5 | μM |
| dNTPs Mixture (each of 2.5 mM) | 4 | μL | 200 | μM |
| Template DNA | 10 | ng | cDNA <1 μg; | |
| | | | plasmid <10 ng | |
| PrimeSTAR HS DNA Polymerase | 0.5 | μL | 0.02 | U/μL |

| | |
|---|---|
| (Takara, 2.5 U/μl) | |
| ddH₂O | to 50 μL |

Wherein the sequence of the forward primer is: 5′-gacgtcagacgeggaagcttcgatc-3′ (SEQ ID NO: 9) and the sequence of the reverse primer sequence is: 5′-gctgtttaaacgcccgggctgtag-3′ (SEQ ID NO: 10).

The procedure for PCR amplification is seen in Table 2:

TABLE 2

| Step | Temperature | Time | |
|---|---|---|---|
| Denaturation | 98° C. | 180 | seconds |
| 34 cycles | 98° C. | 10 | seconds |
| | 62° C. | 15 | seconds |
| | 72° C. | 160 | seconds |
| Final extension | 72° C. | 5 | minutes |

| | | |
|---|---|---|
| Hold | 4° C. | Holding |

2) Electrophoresis was conducted on 1% Agarose gel (Genescript) using the corresponding DNA makers (Takara) as a control, to confirm the correctness of PCR-products. The band expected at the position of about 2.2K bp in the gel was cut out and recovered for the retained PCR product with reference to the instructions, using QIAquick Gel Extraction Kit (QIAGEN). The concentration of the product was determined with Nano-300 to be 86 ng/μl.
3) The pRC vector was subjected to double restriction digestion (restriction digestion at 37° C. for 2 h) according to the reaction system as shown in Table 3 below:

TABLE 3

| Constituents | Reaction system |
|---|---|
| (purchased from NEB) | of 50 μL |
| 10 × CutSmart buffer | 5 μL |
| pRC vector | 2 μL (2 μg) |
| Hind III-HF | 1 μL |
| Xma I | 1 μL |
| H₂O | 41 μL |

4) Electrophoresis was conducted with 1% agarose using the corresponding DNA makers as a control, to confirm the correctness of restrictively digested products. The band expected at the position of about 2.2K bp in the gel was cut out and recovered for the rest of PCR products with reference to the instructions of QIAquick Gel Extraction Kit. The concentration of the product was determined with Nano-300 to be 20 ng/μl.
5) The product purified above was subjected to ligation according to the reaction system as shown in Table 4 below (ligation at 50° C. for 1 h):

TABLE 4

| | Reaction system of 10 μL | |
|---|---|---|
| Constituents | Ligation group | Control group |
| MML assembly mix (purchased | 5 μL | 5 μL |
| from Shanghai Langjing) | | |
| pRC (linearized) | 2 μL | 2 μL |
| Restriction digested amplification | 1 μL | — |
| product of RC-C08/C15/C18 | | |
| serotype Cap | | |
| ddH₂O | 2 μL | 3 μL |

6) The ligation product was transformed into Stb13 competent cells with reference to the instructions of Stb13 Chemically Competent Cell (WEIDI).
7) Single colonies on LB (Kana) plates were picked into sterile 1.5 mL tubes filled in advance with 200 μl LB (Kana) medium, cultured at 37° C. under 250 rpm for 3 h, and subjected to Colony PCR to screen positive clones according to the reaction system as shown in Table 5 below:

TABLE 5

| | Reaction system of 20 μL | |
|---|---|---|
| | Screening | Blank |
| Constituents | with PCR | control |
| Premix Taq Takara Taq ™ Version | 10 μL | 10 μL |
| 2.0 plus dye (Takara) | | |
| C08-WF primer (10 μM) (synthesized | 1 μL | 1 μL |
| by the trustee Shanghai Westech | | |
| Biotechnology, with the primer | | |
| sequence of 5′-atggctgccgatggttatct-3′) | | |
| M13F primer (10 μM) (purchased from | 1 μL | 1 μL |
| Shanghai Westech Biotechnology) | | |
| Bacterial liquid | 2 μL | — |
| ddH₂O | 6 μL | 8 μL |

The procedure for Colony PCR is shown in Table 6:

TABLE 6

| Step | Temperature | Time | |
|---|---|---|---|
| Initial denaturation | 94° C. | 3 | minutes |
| 34 cycles | 94° C. | 30 | seconds |
| | 55° C. | 30 | seconds |
| | 72° C. | 45 | seconds |
| Final extension | 72° C. | 5 | minutes |

| | | |
|---|---|---|
| Hold | 4° C. | Holding |

8) Identification with agarose gel electrophoresis Three recombinant clones are confirmed with PCR of bacterial liquid (5 individual colonies selected for each clone), and all of the colonies are confirmed to be positive.
9) The clones positive under bacterial test (#1 #2 #3) are selected for sequencing.
10) Once the sequencing results are aligned to be correct, plasmids are extracted according to the instructions using TIANGEN EndoFree Maxi Plasmid Kit (TIANGEN DP117).
11) After extraction, the plasmid was subjected to restriction digestion for identification (restriction digestion at 37° C. for 1 h) with the two Bam HI sites according to the reaction system as shown in Table 7 below:

TABLE 7

| Constituents | Reaction system of 20 μL | |
|---|---|---|
| 10 × CutSmart buffer | 2 | μL |
| Plasmid | 1 | μg |
| Bam HI | 1 | μL |

| | |
|---|---|
| ddH₂O | to 20 μL |

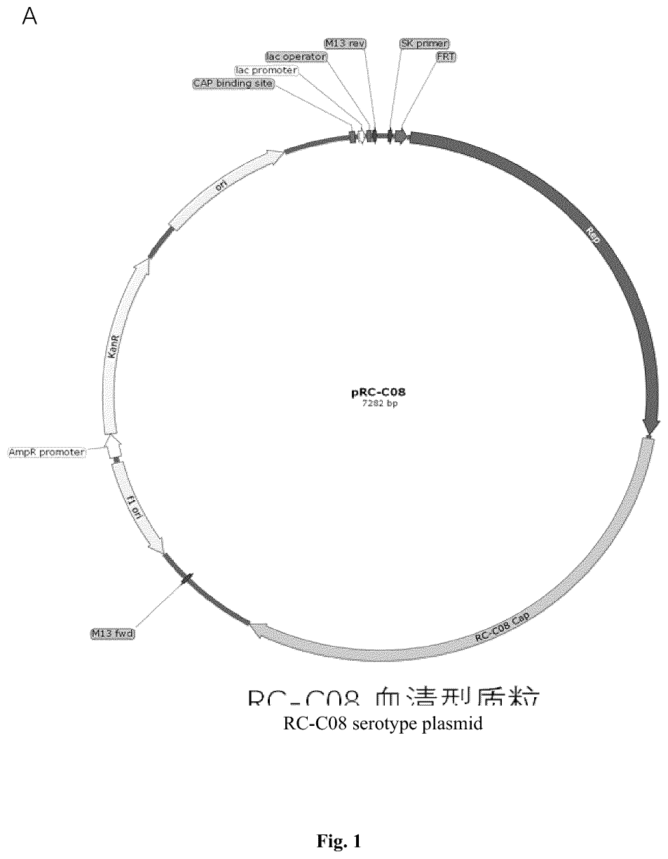

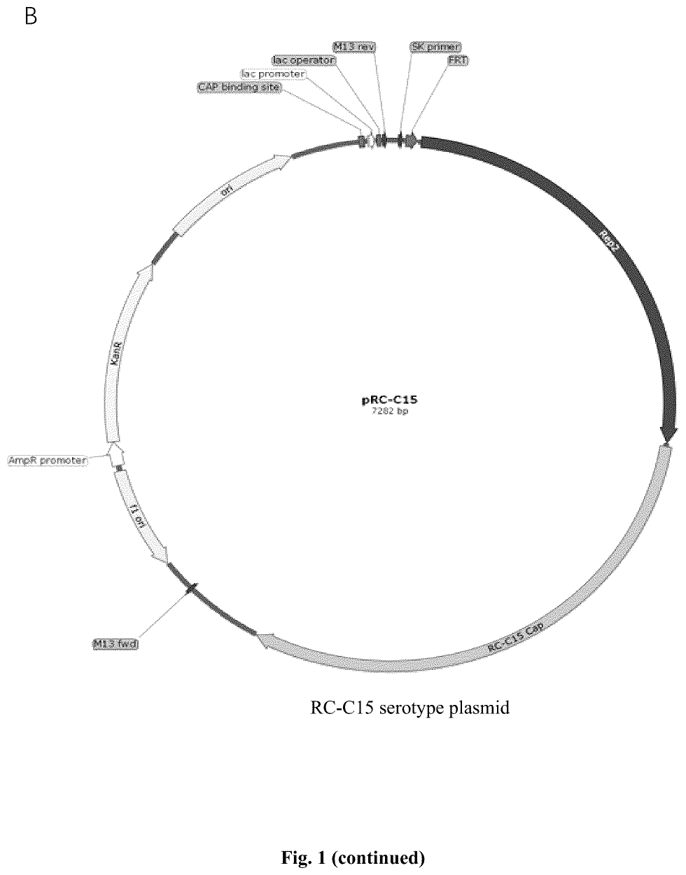

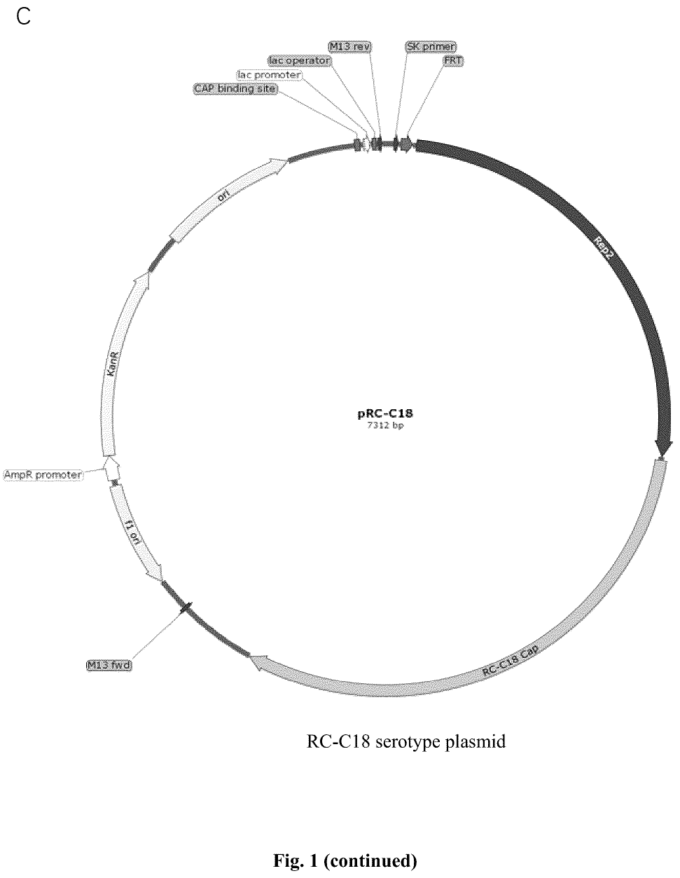

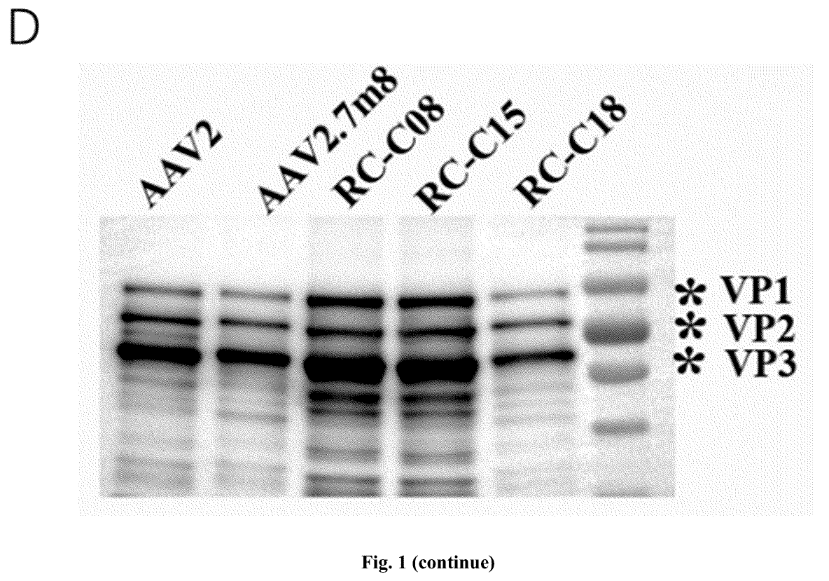

The sequencing result demonstrates that Cap gene sequences of RC-C08/RC-C15/RC-C18 serotypes of the present invention have been successfully constructed into the pRC vectors of AAV2, respectively, yielding the plasmids for the three serotypes which are designated pRC-C08, pRC-C15 and pRC-C18 (the plasmid maps drawn with Snapgene are seen in FIGS. 1A, 1B and 1C, respectively).

Subsequently, the present inventors performed viral packaging with three serotype plasmids newly constructed above and detected in Western blotting (denaturing gel electrophoresis) the size and the proportions of the compositions of the expressed capsid proteins of the 5 serotypes abovementioned, using VP1-specific antibodies which can detect three proteins of the AAV2 capsid in the conventional sense, namely VP1, VP2, and VP3. At the same time, the packaged wild-type AAV2 and AAV2.7m8 viruses were used as the experimental control group (both or either was likewise used in the subsequent experiments as a control group. See below for details). Detection results are shown in FIG. 1D, in which it can seen that, for the three serotypes with engineered capsid proteins, RC-C08, RC-C15 and RC-C18, the molecular-weight sizes of their capsids and the proportions of the three constituent proteins thereof (VP1: VP2:VP3) have no significant difference in comparison with those of the existing serotypes (wild-type AAV2 and AAV2 7m8).

### Example 2: Comparison Between RC-C08, RC-C15, RC-C18 and the Existing Serotypes in the Viral

### Yielding Efficiency

### Procedure for Viral Packaging (in Adherent Cells):

The first step is the cell passage: HEK-293T cells in a 10-cm dish are passaged at 1:3 when they reach 90% confluence (transfecting 293 suspension cells with plasmids and packaging can be performed only when a cell density of 5E6/ml is reached), and then the plasmid transfection can be performed when the cells reach 80% confluence after 24 h of cultivation. The second step is the preparation of a transfection system: a transfection mixture for each 10-cm dish is prepared according to the following system: 500 μl of serum-reduced medium Opti-MEM (Gibco), 15 μg of HLP plasmid (synthesized by Genescript), 7.5 μg of RC plasmid, 7.5 μg of GOI plasmid, 22.5 μl of PEIpro (Polyplus). The third step is the transfection: add the transfection mixture dropwise into various areas of the 10-cm dish, and then gently cross-shaking to mix well; The fourth step is the packaging and cultivation: the transfected cells is transferred to an incubator with carbon dioxide and cultured at 37 degrees Celsius for 72 h. The last step is the harvest of virus: after 72 h of transfection, the cells are piptetted up with the supernatant on the cells and centrifuged to collect cellular pellets; a lysis solution is then added and the pellet is lysed in a shaking incubator at 37 degrees Celsius for 1 h, centrifuged at 4000 rpm in a swing-rotor for 10 min; the supernatant is collected, filtered with a 0.45 μm needle filter and then aliquoted in a volume of 10 μl for detection of viral yield.

Procedure for packaging AAVs in 293F suspension cells is: 1. after cultured for 2-4 days, 293F suspension cells are sampled and counted for the cell density and viability. When the cell density is greater than 5×10⁶ cells/ml and cell viability greater than 90%, the cells are split for passage, i.e., diluted with the fresh Dynamis™ Medium (Gibco) pre-warmed in a water bath at 37° C. and kept at a cell density of N×10⁵ cells/ml (N ranges from 5-9). The diluted cells are put into a thermostatic shaker at 37° C. with 5% CO₂ at 120 rpm and cultured; Cells to be transfected should be passaged for at least 4 generations after revival and be in the logarithmic growth phase, with a viability of more than 90% and a density of about 2×10⁶ cells/mL. On the day of transfection, cells are sampled and counted for the density and viability. Preparing the transfection solution with the fresh Dynamis™ Medium (Gibco) pre-warmed in a water bath at 37° C. (30 mL of the system): (1) 60 μg of plasmids (1×10⁶ cells/μg) is added at the ratio (helper plasmid:capsid plasmid:GOI plasmid=2:1:0.3) into 1.5 mL of Dynamis™ □edium; (2) 60 μL of Fecto VIR-AAV transfection reagent (PolyPlus) is added into the plasmid diluent, immediately shaked on a vortexer for 3 seconds to mix well, and left to stand at room temperature for 30 min. The transfection solution is added dropwise to a cell culture fluid into which can be added antibiotics 300 μl of PS (the final concentration up to 1%) and 30 μl of 10% P188 (the final concentration of 0.01%), and the culture flask is placed back into a shaking incubator at 37° C. with 5% CO₂ at 120 rpm to proceed with cultivation. Cells are lysed after 72 h of transfection, the way in which cell samples are processed is kept consistent with that for adherent cells, and copy numbers of viral genes (vg) are detected by real-time quantitative PCR.

A gene of interest (GOI) plasmid used in the packaging process is an EGFP plasmid which contains a CAG-promoter-driven gene encoding a green flourescence protein EGFP as the flourescence reporter gene and further includes two ITR sequences each flanking at either side of the EGFP-encoding gene, hence providing the genome of the rAAV vector.

### Procedure for Titration of AAV Virus:

Preparing the Standard: Select a unique restriction site in the GOI plasmid. The linearized DNA is recovered from the cutout gel piece after restriction digestion and agarose gel electrophoresis. The DNA concentration of the recovered product is determined by Nanodrop (Thermo Company) and the copy number is calculated according to the equation: c (copy/μl)=plasmid concentration (ng/μl)*(1e⁻⁹)*Avogadro's number/(660 g/mol* the base pair numbers of the plasmid). The plasmid is diluted to 1e⁻⁹ copies/μl and then aliquoted for cryopreservation at −80° C.

Diluting the Standard: One vial of the aliquoted standard is taken and serially diluted with ddH₂O into 1e8, 1e7, 1e6, 1e5, 1e4, 1e3, and 1e2 copies/μl, serving for the template for the standards.

Preparing and Diluting the Sample: The admixture is prepared according to the following system: Benzonase (Merk) 2.5U, MgCl₂ 2 mM (final concentration), and virus suspension of 5 μl are added, brought up to 49 μl with ddH₂O, gently mixed well and centrifuged, followed by treatment at 37° C. for 1 h, at 85° C. for 20 min; then 1 μl of 10 mg/ml proteinase K (Merk) is added, gently shaked and mixed well, centrifuged, followed by treatment at 55 degrees for 10 min, at 85 degrees for 20 min; at last, the finally treated sample (the 10-fold diluted samples) are further diluted by 100-fold and 500-fold, which gives rise to 1000-fold and 5000-fold diluted samples for the template to be assayed.

### Q-PCR Reaction and Method for Calculating the Titer:

Preparing the System (20 μl): 2X Probe Premix of 10 μl, 10 uM hGHpA-F primer (5′-CACAATCTTGGCTCACTG-3′ (SEQ ID NO: 11)) and hGHpA-R primer (5′-CTGGAATCCCAACAACTC-3′ (SEQ ID NO:12)) each of 0.4 μl, hGHpA (5′-TTCAAGCGATTCTCCTGCCTC-3′ (SEQ ID NO: 13)) probe primer of 0.8 μl, 50X ROX II (Takara) of 0.4 μl, the template of 2 μl, brought up with water to 20 μl; Detection is competed according to the following program: 95° C. for 5 min; followed by 40 cycles of 95° C. for 5 s, 60° C. for 30 s. Data is exported and the titer is calculated as “titer (VG/ml)=the exported data* dilution factor *1000”;

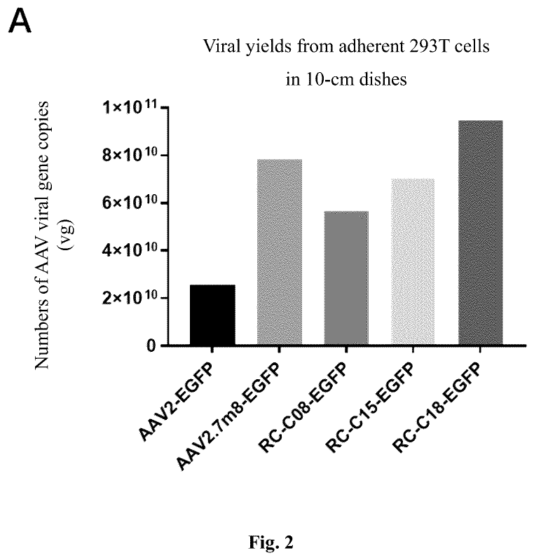

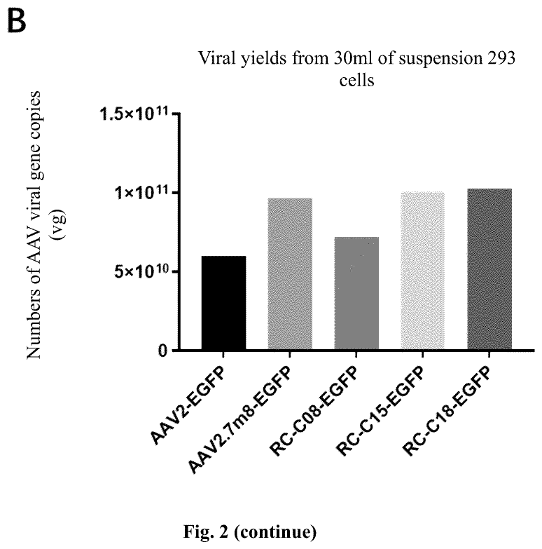

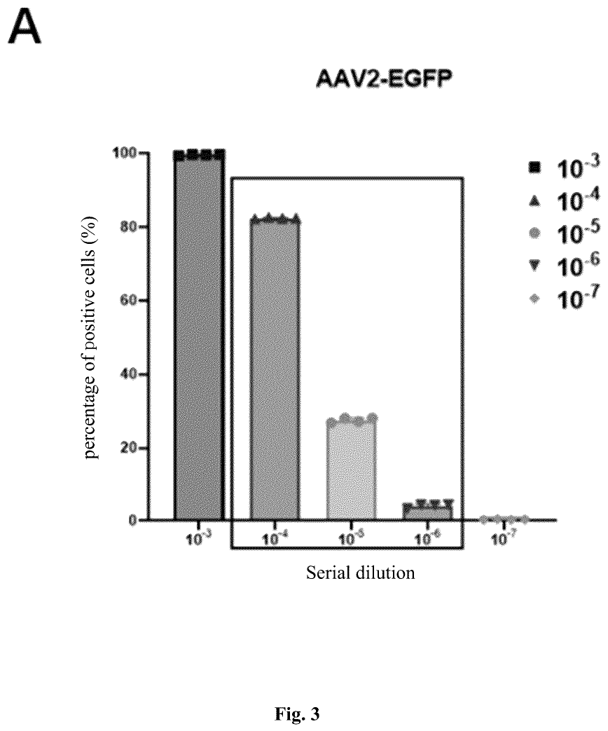

As shown in FIG. 2: viral packaging of the 5 serotypes was conducted in adherent 293 cells and suspension 293T cells, and viral yields were tested for different serotypes. It can be seen from FIG. 2A that differences in viral yields for packaging of AAV2.7m8, RC-C08, RC-C15, and RC-C18 in adherent cells are varied within 5-fold compared to that of wild-type AAV2 virus. Thus, RC-C08, RC-C15, and RC-C18 of the present invention have no significant difference in viral yield compared to the serotype AAV2.7m8 having the targeting property. In the results illustrated in FIG. 2B, when the five viruses abovementioned are packaged in suspension 293 cells using the same packing method of triple-plasmid PEI-based transient transfection, the yields of the resulting five viruses exhibit differences of less than 2-fold and of no statistical significance, although showing a trend in difference similar to those in 293T cells (FIG. 2A).

### Example 3: Comparison of the In Vitro Bioactivity of the Novel Serotypes RC-C08, RC-C15 and RC-C18

the Steps of Determining TUs of the AAV Viruses with Flow Cytometery are as Follows:
1. Preparing adherently cultured 293T cells to be infected and the purified viruses of the five serotypes AAV2, AAV2.7m8, RC-C08, RC-C15, and RC-C18.
2. Day1. Plating the cells: after being detached by digestion, HEK293T cells are collected by centrifugation at 1000 rpm for 5 min, re-suspended, counted, and plated in the wells of a 96-well plate at a density of 1E+4 HEK293T cells/well.
3. Day2. Infected with the Virus: the cells are counted after being plated for 24 h;

The AAVs are diluted serially with a complete culture medium, i.e., sequentially diluted 10-fold in the following dilutions:

$$\begin{matrix}1 \times 1 {0}^{- 2} = 990 μL of diluent + 10 μL of stock \\ 1 \times 1 {0}^{- 3} = 900 μL of diluent + 100 μL of previous dilution \\ 1 \times 1 {0}^{- 4} = 900 μL of diluent + 100 μL of previous dilution \\ 1 \times 1 {0}^{- 5} = 900 μL of diluent + 100 μL of previous dilution \\ 1 \times 1 {0}^{- 6} = 900 μL of diluent + 100 μL of previous dilution \\ 1 \times 1 {0}^{- 7} = 900 μL of diluent + 100 μL of previous dilution\end{matrix}$$

The overnight cultured cells are taken out and the complete culture medium is pipetted out from the wells, followed by addition of 100 μL of the medium containing the serially diluted viruses (8 wells for each dilution): place into a cell incubator at 37° C. with 5% CO₂ for 72 hours of cultivation.
5. Day5. Take pictures of the digested cells: 72 hours later, the medium is discarded. 100 μL of PBS is added to the cells for washing and then discarded. 20 μL of Trypsin is added and the cells are placed in an incubator at 37° C. for 3 min of digestion. The digestion is stopped by adding 80 μL of DMEM containing 10% FBS (FBS is purchased from Gibco and DMEM is purchased from Hyclone). Viral fluorescence is detected with flow cytometery.
6. Data is exported. The result of flow cytometery is analyzed and processed with FlowJo V10, generating an Excel file which is converted into histogram plots with Graphpad Prism9.

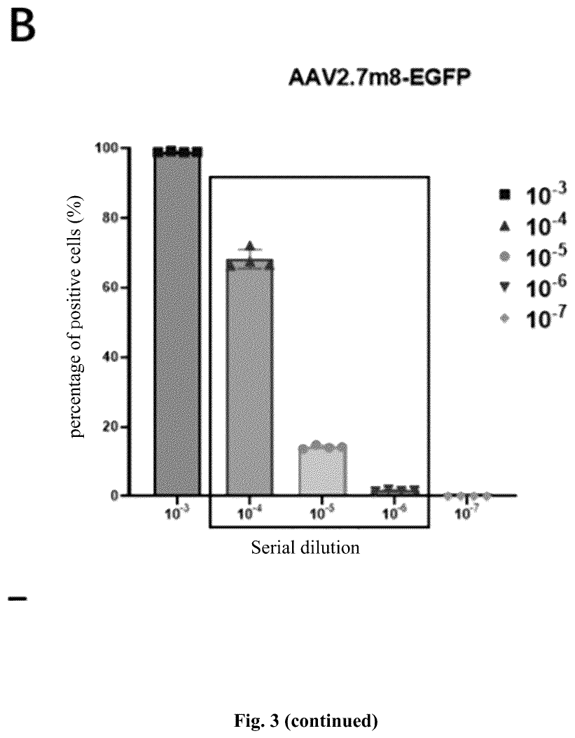

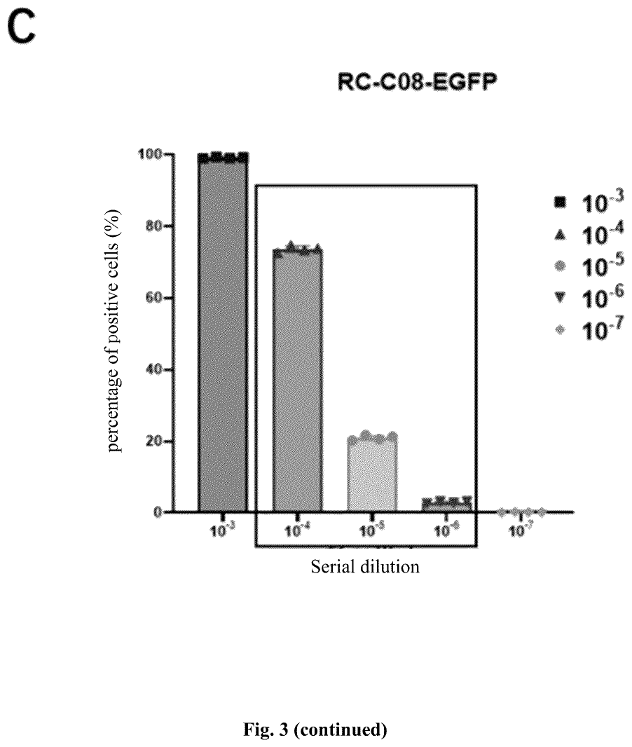

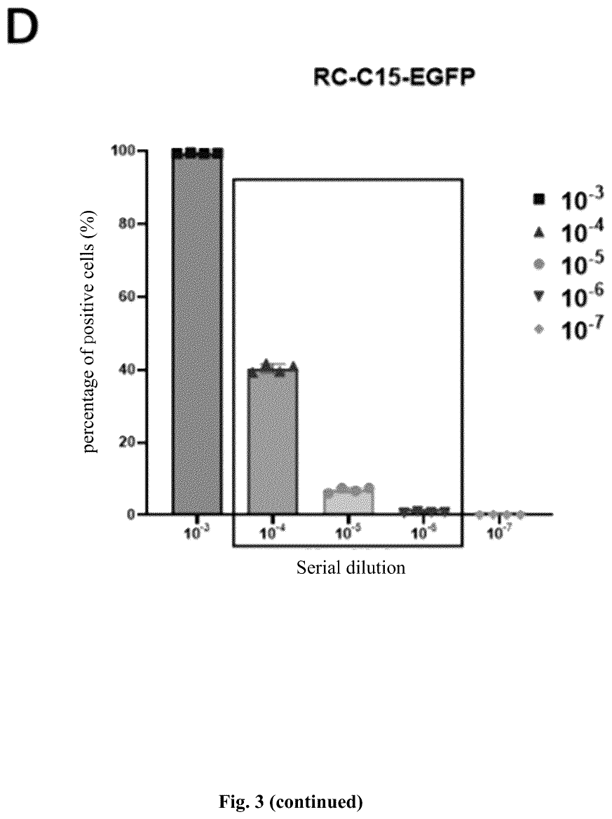

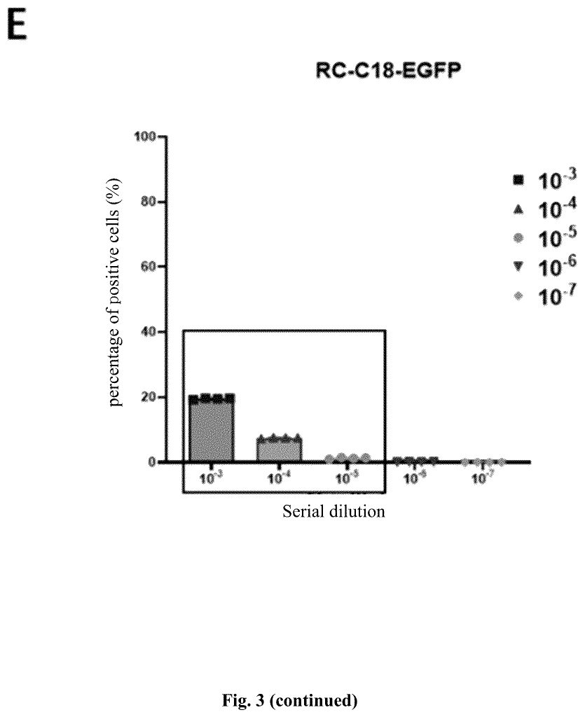

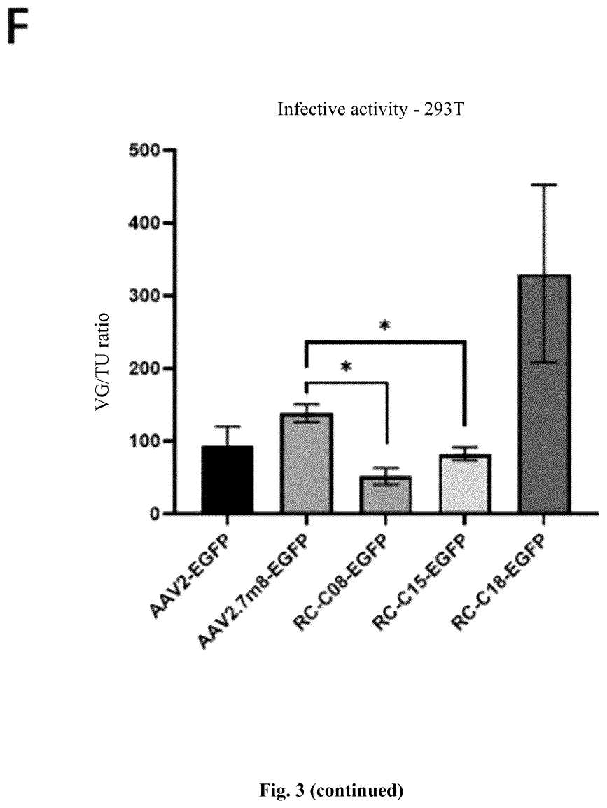

Analysis of the Results: As shown in FIG. 3, TUs of the viruses RC-C08 and RC-C15 approximate to those of AAV2 and AAV2.7m8, while infective activities thereof detected in 293T are all significantly superior to that of RC-C18 (FIGS. 3A-3E), wherein RC-C08 has the lowest VG/TU value (with the same vg, the lower the value is, the stronger the in vitro transduction activity of the virus is). RC-C08 has the strongest in vitro transduction activity and is followed by RC-C15, as compared to the currently known serotypes such as AAV2, AAV2.7m8, and the like. Activities of the RC-C18 mutant serotypes are weakest in 293T, compare to AAV2.7m8 (FIG. 3F). The results depicted above demonstrates that the RC-C08 of the present invention has significant advantage superiority in the in vitro transduction activity as compared to the wild-type AAV2 serotype.

### Example 4: Comparison of In Vitro Transduction Efficiency Between RC-C08 and the Three Existing Serotypes AAV2, AAV2.7m8, AAV-DJ

The procedure for determining in vitro transduction activities of AAV viruses is as follows:

### Procedure for Determining Viral Infection Capacity

1. Cells Four cell types: HEK293T (ATCC; CRL-11268), CHO (ATCC; CRL-2092), ARPE19 (ATCC; CRL-2302), and 661w (Lonza). Among them, 661W is a photoreceptor cell line from mouse and the photoreceptor cell lines of human origin can't be designed into the experiment due to scarce availability.
2. Virus: Four viral serotypes: AAV2, AAV2.7m8, AAV-DJ, and RC-C08.
3. Multiplicity of Infection Established for In vitro Infection with Virus: HEK293T MOI=200, CHO MOI=2000, 661w MOI=200, ARPE19 MOI=1000.
4. Day1. Plating the Cells: the cells of HEK293T, 661w, CHO, and ARPE19 in culture are detached with trypsin digestion, centrifuged at 1000 rpm for 5 min, collected, counted, plated into 96-well plates in the following number of cells per well: HEK293T 1E+4, CHO 1E+4, 661w 5E+3, ARPE19 1E+4.
5. Day2. Infected with the Virus: the cells are counted after being plated for 24 h and the desired MOI of the virus is added on the basis of the counted number.
6. Day5. Take Photos of the Digested Cells: After the medium is discarded, 100 μL of PBS is added to the cells for washing and then discarded. 20 μL of Trypsin (Gibco) is added and the cells are placed in an incubator at 37° C. for digestion. After the digestion is performed completely, it is stopped by adding 80 μL of DMEM containing 10% FBS. Cells are pipetted up and down to archive uniformity. Viral fluorescence is detected with flow cytometery.
7. Data is exported. The result of flow cytometery is analyzed and processed with FlowJo, generating an Excel file which is converted into histogram plots with graphpad.

Both the fluorescence percentage and the mean fluorescence intensity (MFI) of the RC-C08 group in HEK293T, 661w and CHO are significantly higher than those of the AAV2, AAV2.7m8, and AAV-DJ groups. The fluorescence percentage and the mean fluorescence intensity (MFI) of the AAV2.7m8 group in ARPE19 cells are significantly higher than those of the AAV2, AAV-DJ, and RC-C08 groups.

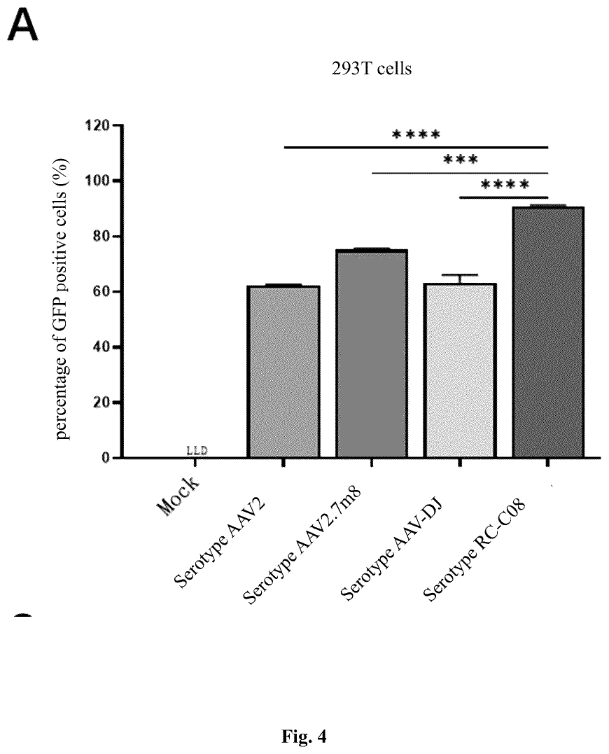

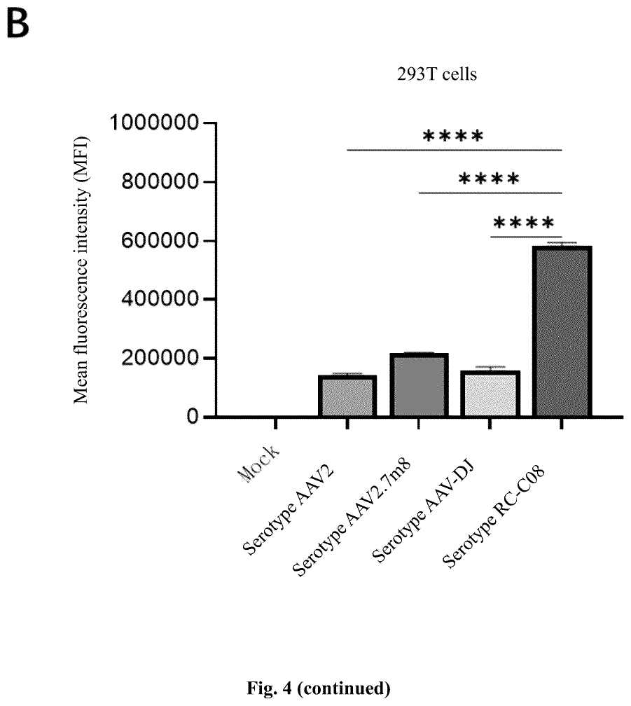

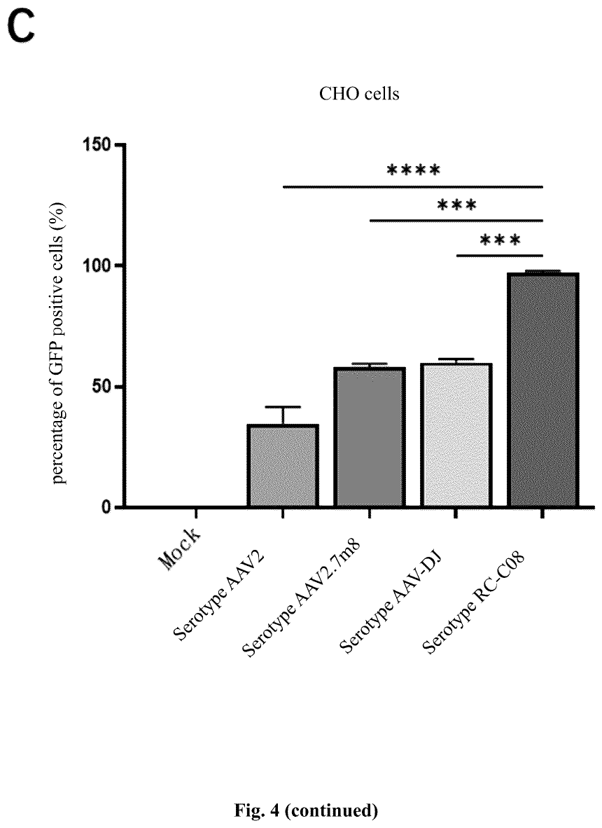

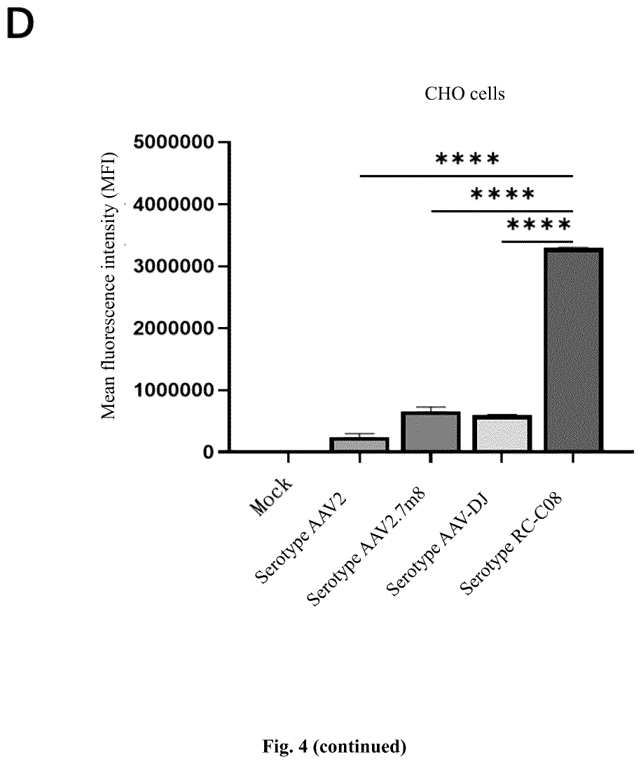

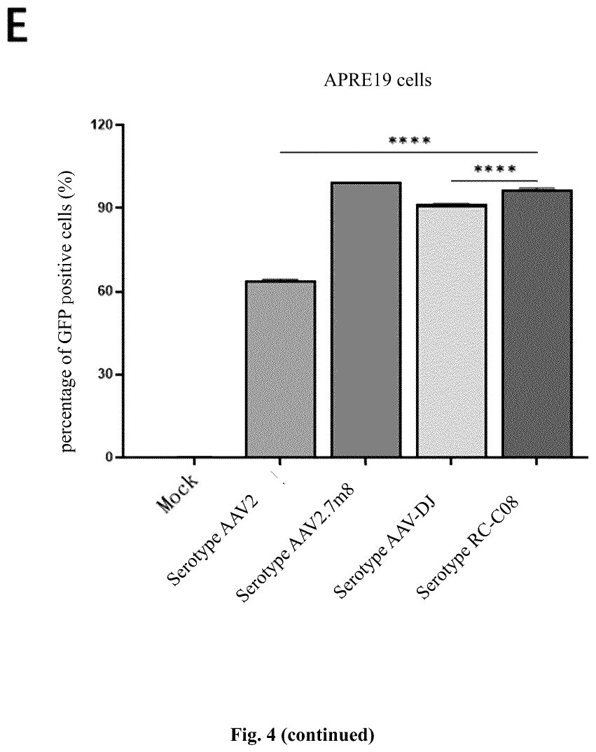

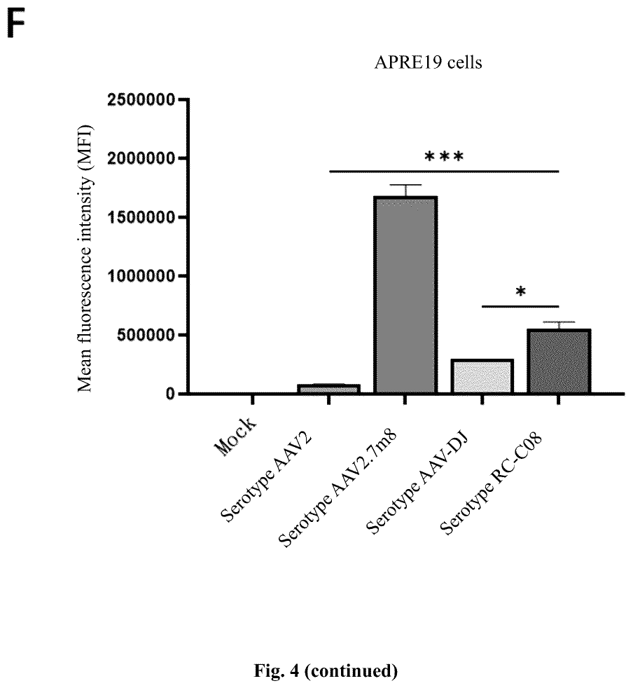

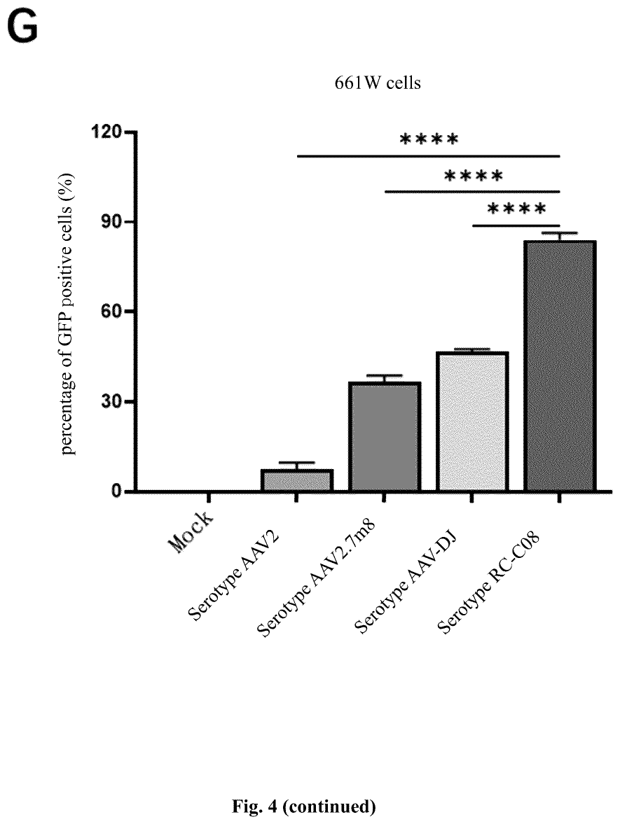

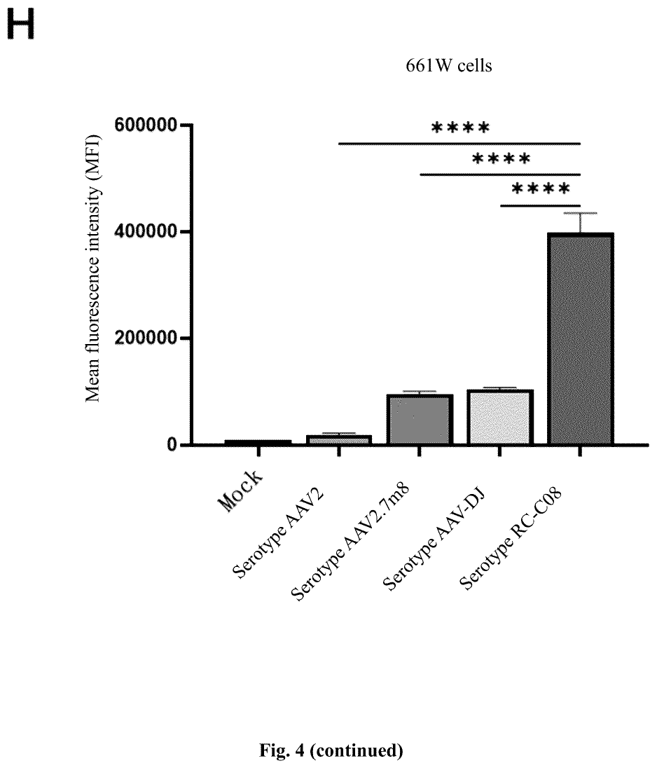

According to the characteristics documented in the currently published literature, the AAV-DJ serotype has the strongest transduction activity in vitro among the existing serotypes, especially in 293T cells. Hence, we have compared the in vitro infection efficiencies in different cells between the serotype RC-C08 and the serotypes AAV2 (wild type), AAV2.7m8, AAV-DJ. The results indicate that, as shown in FIG. 4, the statistical results from FIGS. 4A-4D show that the GFP fluorescence percentage and mean fluorescence intensity (MFI) of RC-C08 in HEK293T and CHO cells are significantly higher than those in the AAV2, AAV2.7m8, and AAV-DJ groups. Moreover, since the same fluorescence reporter system (EGFP) is used in all of the four recombinant viruses, the MFI values reflect that the mean fluorescence intensity in a single-cell, as detected by FACS in cells infected with RC-C08, is over 3-fold higher than those of the other three viruses, indicating the strongest transduction activity of RC-C08 in 293T and CHO cells. Thus, it can be seen that, as compared to the wild-type AAV and other known variant viruses having high transduction activities in vitro (AAV2.7m8 and AAV-DJ), the novel serotype RC-C08 has a significantly increased cell infection activity in vitro. Similar conclusions were also drawn in mouse photoreceptor cells (661w). As shown in the statistical results of FIGS. 4G-4H, the fluorescence percentage and the mean fluorescence intensity (MFI) of the RC-C08 group were significantly higher than those of the serotypes documented in the prior art such as AAV2, AAV2.7m8, and AAV-DJ. Nonetheless, the statistics on infective activities in the retinal pigment epithelial cells (ARPE19) demonstrates that the numerical values of fluorescence percentage and MFI in the AAV2.7m8 group are significantly higher than those in the AAV2, AAV-DJ and RC-C08 serotype groups (see FIG. 4E-4F). Thus, it can be seen that, compared to the wild-type AAV and other known AAV serotypes having high transduction activities, RC-C08 has the significantly increased transduction activity for photoreceptor cells in the retinal tissue, and the decreased infectivity to pigment epithelium cells such as ARPE19.

### Example 5: Comparison of the Difference in the In Vivo Transduction Efficiency Between the Serotype RC-C08 and the Serotypes AAV2.7m8 & AAV-DJ

Six (6) of C57 WT mice were prepared and randomized according to two mice per group. The prepared serotypes RC-C08-EGFP, AAV2.7m8-EGFP, and AAV-DJ-EGFP were diluted to E11 vg/ml and injected intravitreally (IVT) in a volume of 2 μl into the left (OS) and right (OD) eyes of each mouse, respectively. On Day 40 and Day 60 after administration, the in vivo transduction efficiency was evaluated by intravital auto-fluorescence (AF) and retinal flat mount assays.

The experimental steps in the intravital auto-fluorescence assay (AF) are as follows: C57 mice of 6-8 weeks of age with normal appearance (GemPharmatech Co., Ltd) are checked with ear tags, followed by dropping a mydriatic agent onto the ocular surfaces of both eyes, and are anesthetized with Zoletil mixture (Virbac, BN 7T78) at a dose of 60 mg/kg. A surface anesthetic is dropped onto the ocular surfaces of both eyes, and gel is applied onto the ocular surfaces to wear contact lenses. Set the HRA control panel on the Heidelberg SPECTRALIS optical coherence tomography (OCT) inspection equipment to IR mode, focus on the mouse fundus until the image is clear, then switch to FA mode, adjust the SENS value to 107, adjust the focal length until the retinal blood vessels can be clearly seen, reduce the SENS value to 100, and start taking photos.

The steps of retinal flat mount assay are as follows: Mouse eyeballs are enucleated and then fixed with 4% paraformaldehyde for 30 min. After the end of fixation, the mouse eyeballs are soaked in PBS to wash and remove the residual fixation solution. Under a stereomicroscope (LEICA S9), cut open the mouse eyeball along one side of the margin of the sclera near the sclera. After removing the cornea and lens, hold a pair of tweezers in the left hand to fix the optic nerve, and use another pair of tweezers in the right hand to push the retina out of the optic cup along the optic nerve. Cut the retina along the margin into a petal shape. Transfer the retina onto a glass slide using tweezers and flatten it, then stain the nucleus with DAPI. Drop the anti-fluorescence quenching mounting medium to the retina, and apply a little nail varnish on the edge of the glass slide for fixation. Cover with a glass slide for microscopy. Use the Life EVOS M700 for panoramic scanning and photography of retinal tissue, with green fluorescent channel imaging, automatic stitching and combination.

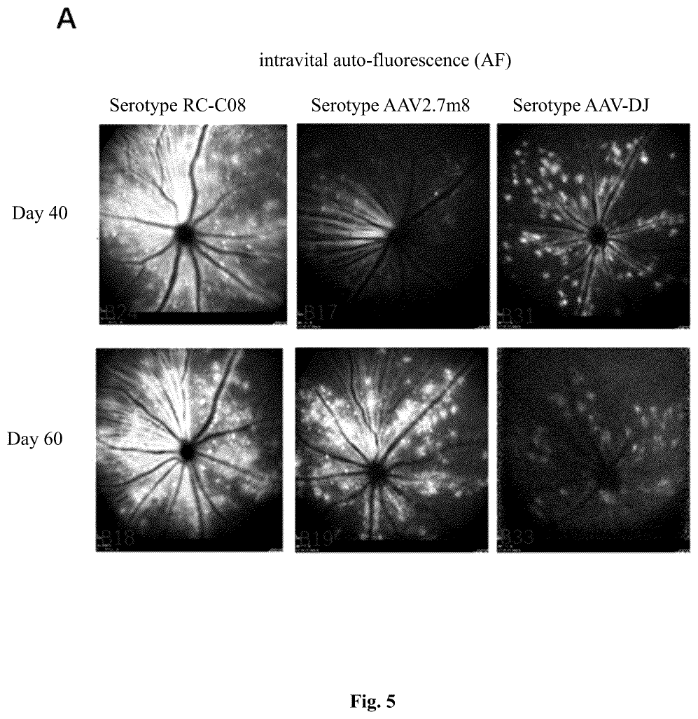

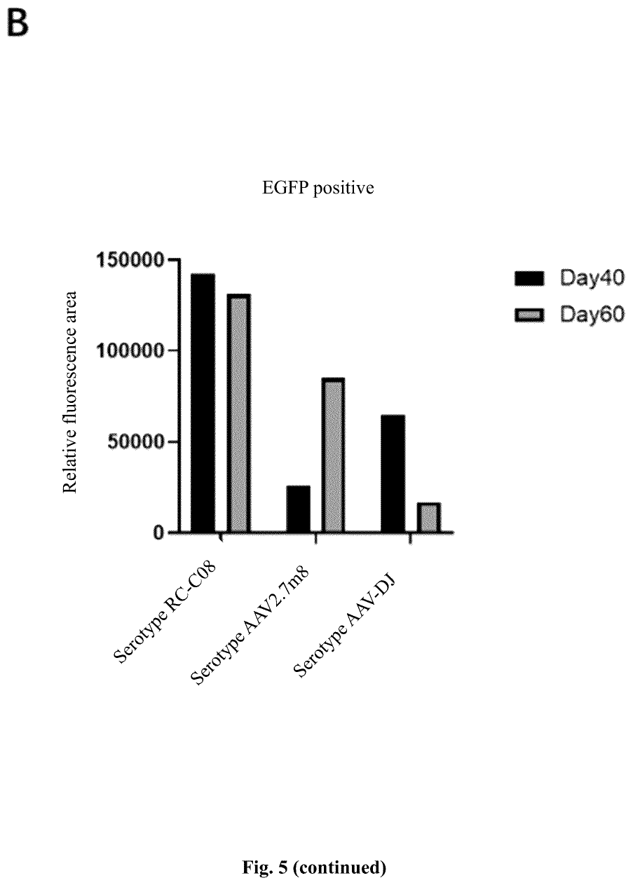

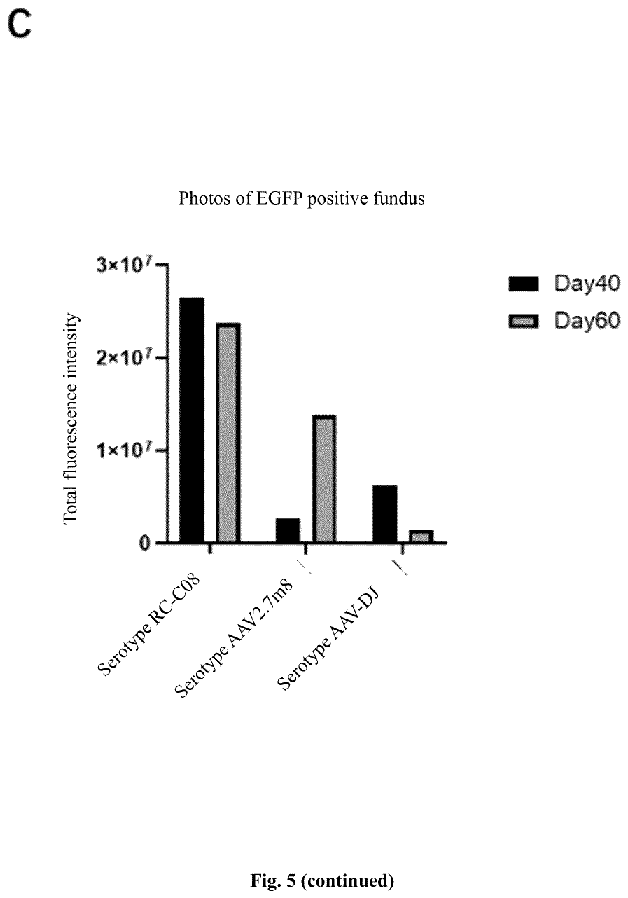

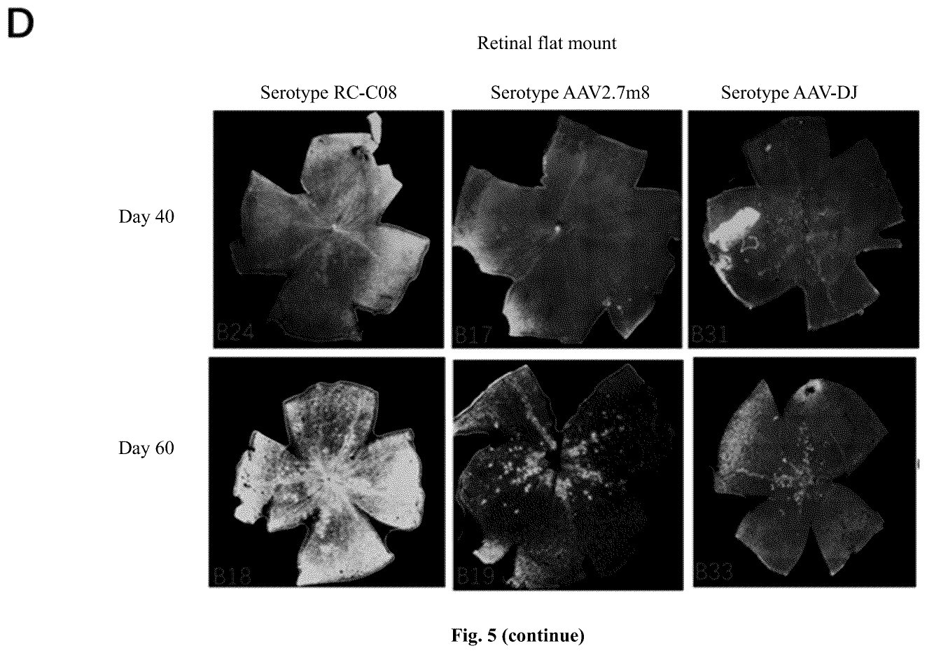

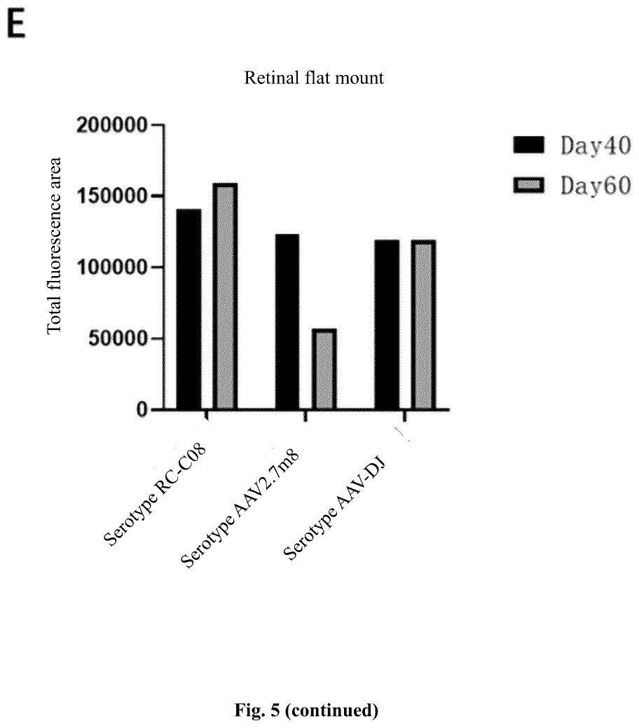

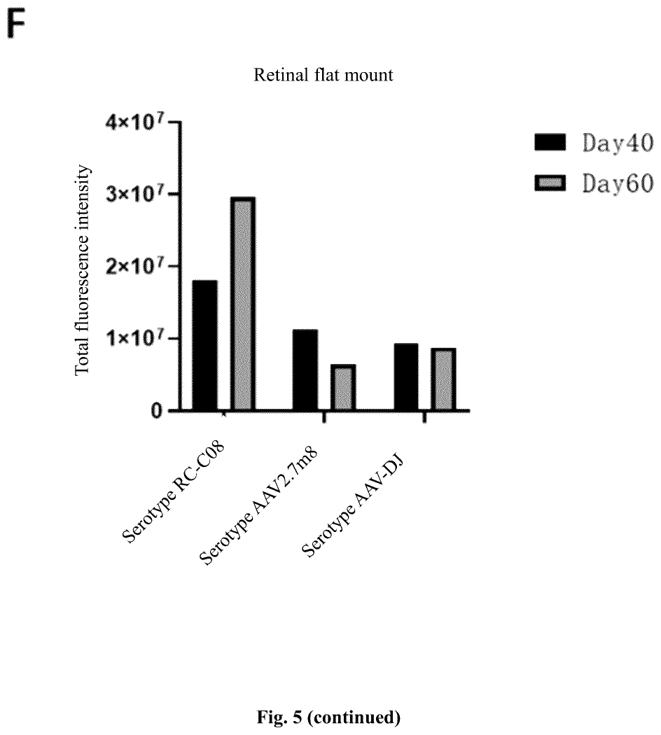

Results of AF assay are shown in FIGS. 5A-5C. According to FIG. 5A, it can be known that all the three serotypes can reach retinochoroidal tissues through IVT administration and express the green fluorescence protein. Further by analyzing the photos of AF assay with ImageJ 1.8.0 grey-scale scanning, the relative fluorescence area of mouse fundus is obtained (FIG. 5B) and the total fluorescence intensity is calculated statistically (FIG. 5C). It can be found from the statistical results that the fluorescence intensity of RC-C08 serotype is significantly higher than those of the control groups (AAV2.7m8 and AAV-DJ serotypes) at 40 and 60 days after IVT administration. It is shown that, as the time went by, the expression of AAV2.7m8 serotype virus in the ocular tissue increased continuously, while the expression intensity of AAV-DJ serotype significantly decreased. However, the expression in the experimental group RC-C08 reached its peak after 40 days of administration, and the total fluorescence intensity on Day 40 was 8-10 times that of the experimental control group. Both the total fluorescence intensity and relative fluorescence area did not decrease over time, consistently maintaining a higher level of sustained expression.

The results of the retinal flat mount are shown in FIGS. 5D-5F. In FIG. 5D, it can be clearly found that both the area and total intensity of fluorescent protein distribution in the retinal tissue 40 and 60 days after IVT administration of RC-C08 virus are significantly higher than those in the control groups (AAV2.7m8 and AAV-DJ serotypes), which is consistent with the phenomenon observed in FIG. 5A. FIGS. 5E and 5F demonstrate the comparison between Day 40 and Day 60. With the time went by after administration, the expression of AAV2.7m8 serotype virus in eye tissue on Day 60 was enhanced to some degree compared to that on Day 40. The expression intensity of the AAV-DJ serotype in the retinal tissue showed a decreasing trend, while the total fluorescence intensity and fluorescence area of RC-C08 serotype of the present invention was significantly increased. The assay results on Day 60 post-administration showed that both the total fluorescence intensity and fluorescence distribution area in the retinal tissue were significantly improved compared to those on Day 40. The comparison between the experimental group and the control groups is essentially consistent with that in FIGS. 5B-5C.

### Example 6: Comparison of the In Vitro Transduction Activity in Different Cell Lines Among the Three Novel Serotypes RC-C08, RC-C15, and RC-C18

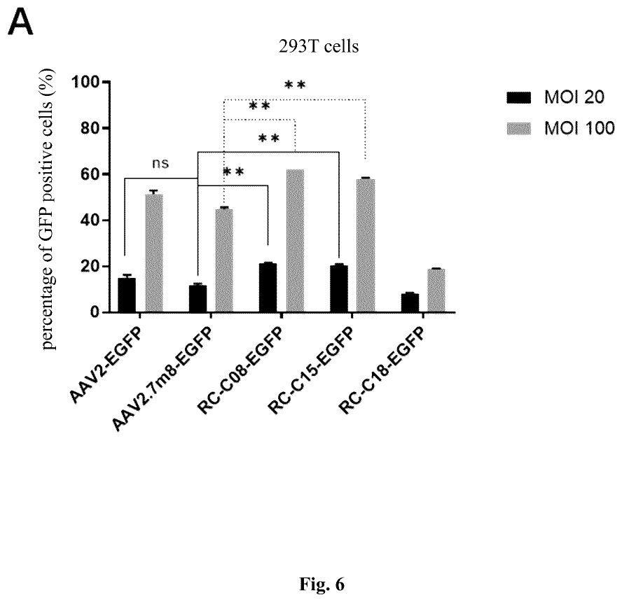

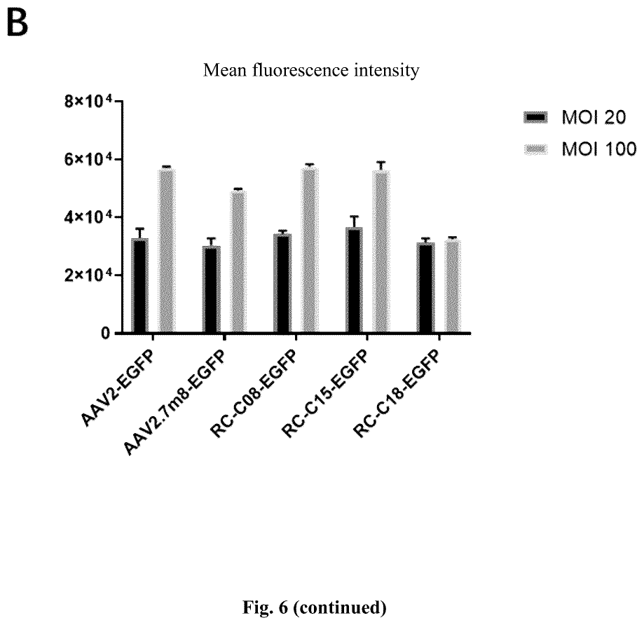

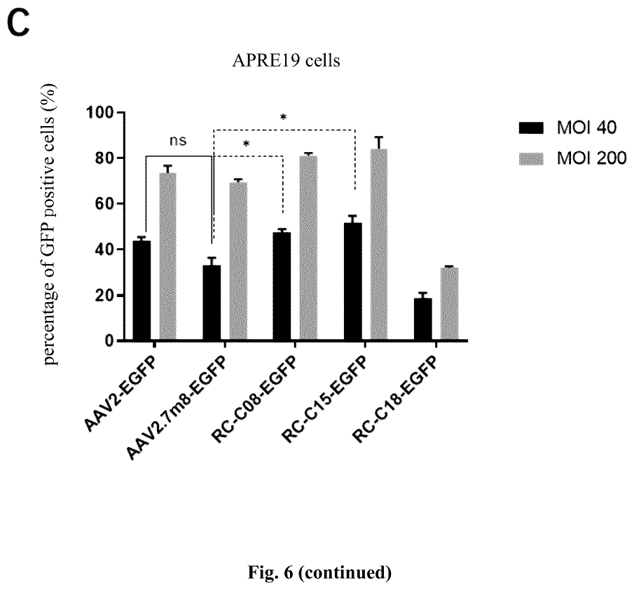

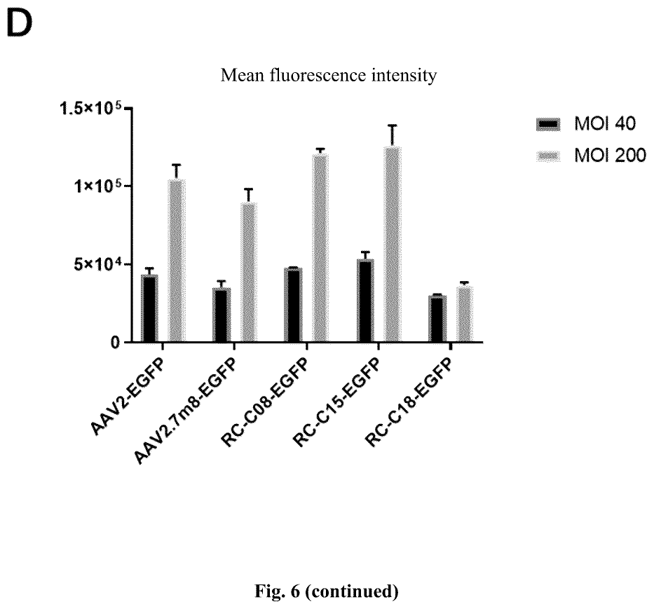

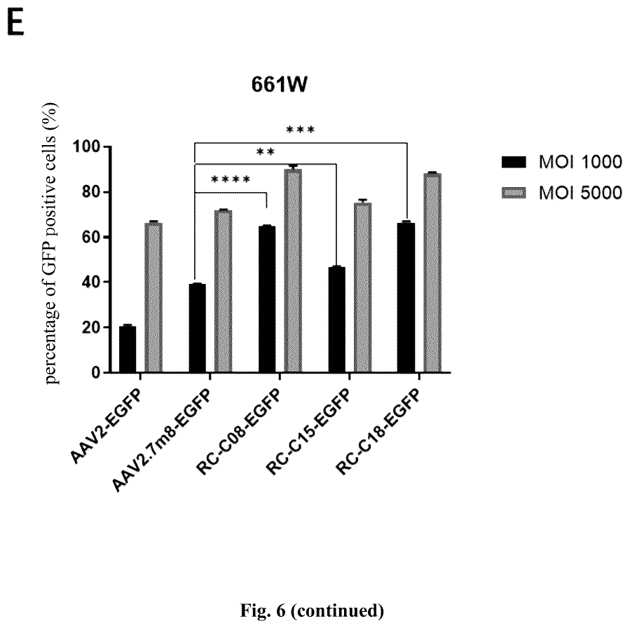

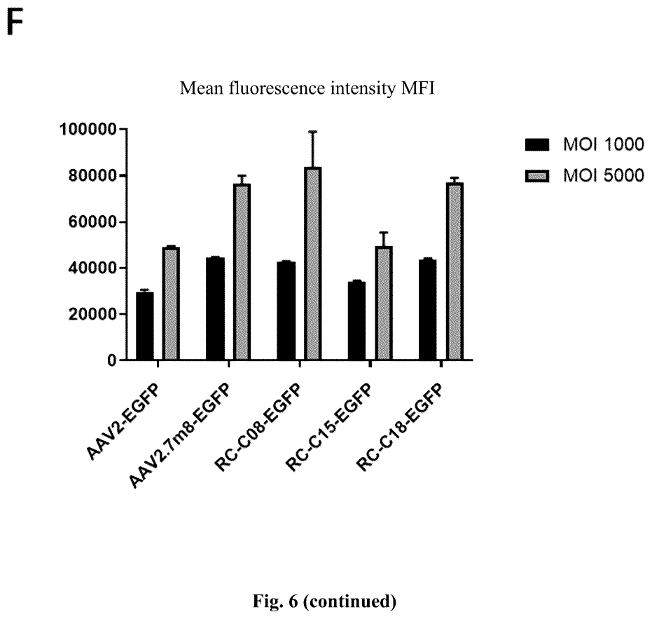

Well-prepared three engineered serotypes of the present invention, RC-C08, RC-C15, and RC-C18, with the gene of interest EGFP inserted therein, were tested for differences in in vitro infective activity, likewise selecting the viruses AAV2 WT and AAV2.7m8 as the experimental control groups. The MOI values were tested in 293T to be 20 and 100, in ARPE19 to be 40 and 200, and in 661W to be 1000 and 5000. Procedures for cultivation and infection thereof are referred to in the Examples as described above. After 72 hours of in vitro infection, the green fluorescence-positive cells were analyzed and detected quantitatively with flow cytometer, and the obtained data were analyzed with the FlowJo official software, resulting in the analytical data as shown in FIGS. 6A-6F.

Analysis of the statistical results showed that the percentage of GFP fluorescence and the mean fluorescence intensity of RC-C08 serotype attained in 293T and ARPE19 cells were significantly higher than those of the control groups AAV2 and AAV2.7m8, while the percentage of GFP fluorescence of RC-C18 serotype attained in 293T and ARPE19 cells was significantly lower than those of AAV2 and AAV2.7m8 (FIGS. 6A-6D). It was also found that the percentages of GFP fluorescence of three variant serotypes RC-C08, RC-C15, and RC-C18 obtained in 661w cells were significantly higher than those of AAV2-EGFP and AAV2.7m8, with the significant differences of p<0.0001, p<0.01, and p<0.001, respectively (FIG. 6E). From this, it can be seen that compared to the serotypes documented in the prior art, RC-C18 exhibits specificity to photoreceptor cells, while the transduction efficiencies of RC-C08 and RC-C15 for photoreceptor cells are significantly different in comparison to that of the AAV2.7m8 serotype.

### Example 7: Validation of the Transduction Activity In Vivo of RC-C08 Serotype and Evaluation of the Short-term Effect of 2-week Intravitreal Administration (IVT)

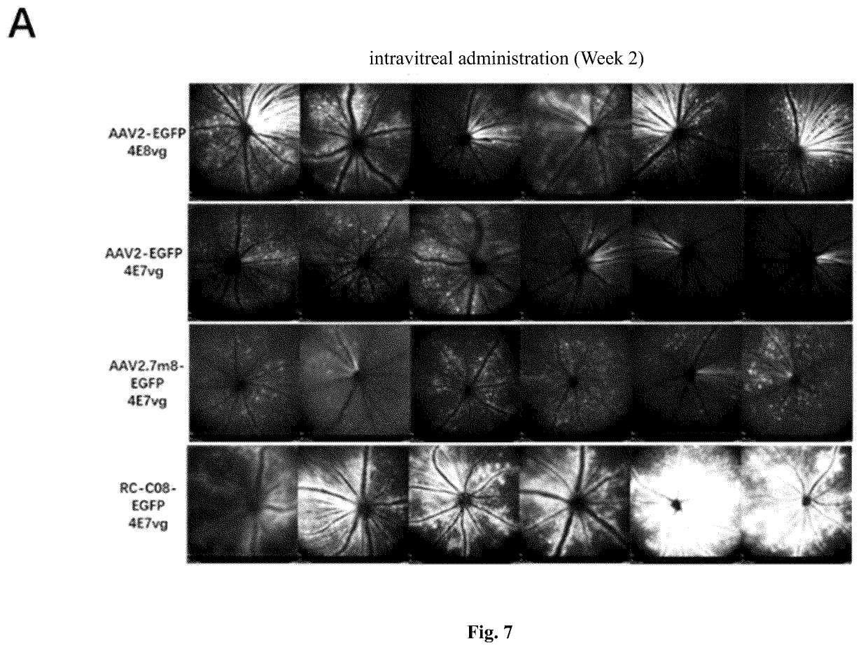

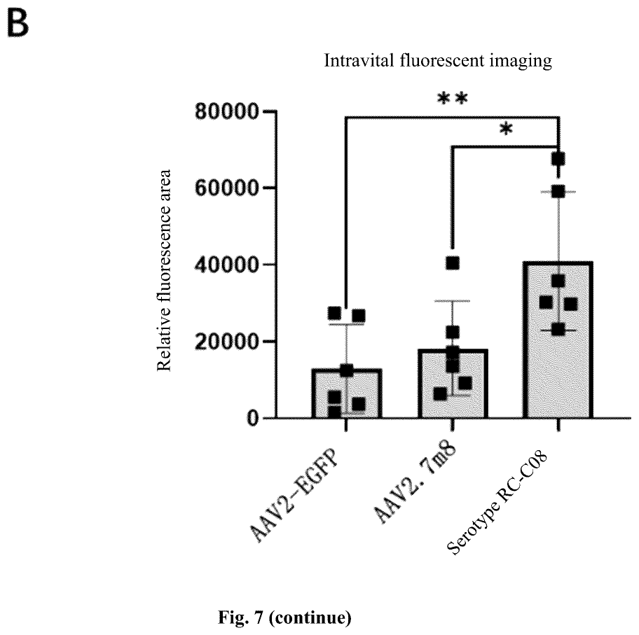

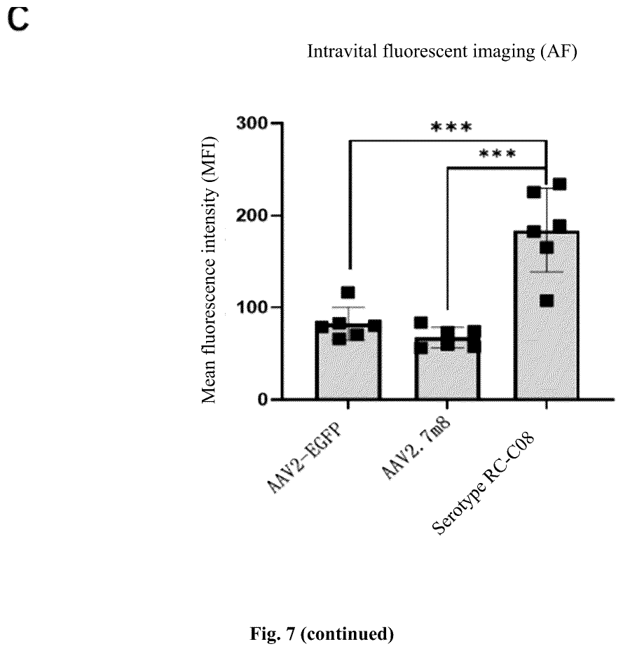

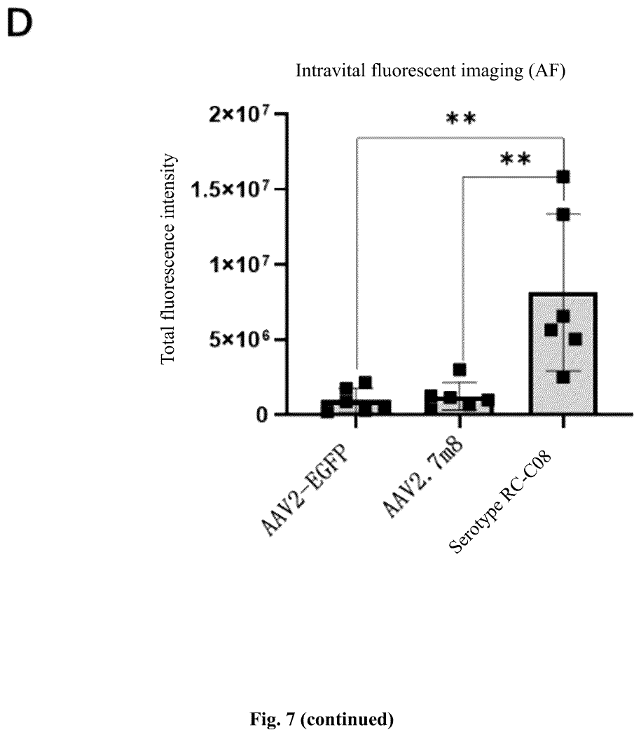

Twelve (12) of C57 wild-type mice were prepare one week in advance and randomized into four groups A-D, namely, the AAV2 control high-dose group, the AAV2 control low-dose group, the AAV2.7m8 control low-dose group, and the RC-C08 experimental low-dose group (see the annotation for dosing regimens in FIG. 7A), with 3 mice assigned to each group. The left and right eyes were administered via intravitreal injection, with 4E8 vg of virus administered in the high-dose group and 4E7 vg in the low-dose group. After 2 weeks of administration, the left and right eyes of all mice in the experimental group were subjected to the intravital auto-fluorescence assay, the procedure of which is referred to the Examples as described above.

As shown in FIG. 7A, it can be observed that when the eyeballs of mice in groups A and D are subjected to the intravital fluorescence assay, fundus autofluorescence signals can be detected. The fluorescent signal of the group D (i.e. the RC-C08 group) is stronger, with strong signals detected in 5 out of 6 eyes. Therefore, as compared with group A (AAV2 high-dose control group), the transduction activity of the RC-C08 dosing group is significantly stronger. Statistics was further performed with the analysis software ImageJ, such as data statistics shown in FIGS. 7B-7C, it is demonstrated from the analytical results of total fluorescence area and mean fluorescence intensity that, the indicators obtained from the RC-C08 serotype is about three times of those of the control groups AAV2 and AAV2.7m8. The total fundus fluorescence intensity was further analyzed and found increased by about 10 times in the RC-C08 low-dose group compared to the control groups (FIG. 7D). It is demonstrated that after 2 weeks of low-dose administration, the transduction activity in vivo of the novel serotype is significantly stronger than that of the existing serotype.

### Example 8: Comparison of the In Vivo Tissue Distribution and Long-Term Activity Between RC-C08 and AAV2.7m8

Six (6) of C57 mice of 6-8 weeks of age purchased from CRO company were divided into two groups A and B, with 3 mice randomized to each group. AAV2.7m8 was administered to Group A, and the RC-C08 serotype was administered into both eyes of 3 mice in Group B, all with the left and right eyes administered via intravitreal injection at a dose of 2E9 vg virus, respectively. After 5 weeks of administration, the intravital auto-fluorescence assay was performed. On Week 6 after administration, the left and right eyes of the 6 mice abovementioned were taken out and frozen sections of eyeball tissues were prepared according to the method documented in Chinese Patent Publication No. CN107012171B. Fluorescence photographs of the retinal tissue were taken at the same exposure intensity and examined.

The steps of the auto-fluorescence assay are referred to the Examples as described above.

The simple operation process for fluorescence photography of frozen sections is as follows: select sections with good morphology of the eyeball slice; soak in and wash with PBS three times, each for 5 min, to remove the OCT embedding solution on the tissue surface; circle the tissue with a histochemical pen and place it horizontally on a humidity box; DAPI is diluted with PBS at 1:2000 and dropped onto the tissue for staining of 5 minutes; soak in and wash with PBS three times, each for 5 min; add anti-fluorescence quenching mounting medium on the eyeball tissue, and apply a little nail varnish on the edge of the glass slide for fixation; cover with a glass slide for microscopy; take photos using the green and blue fluorescent channels, overlap the images, and stitch the small images (10X) together into a complete large image.

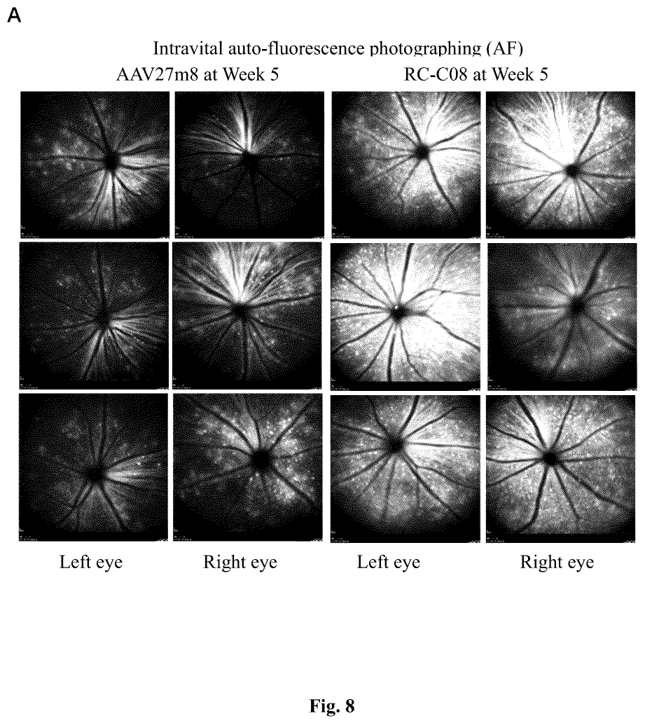

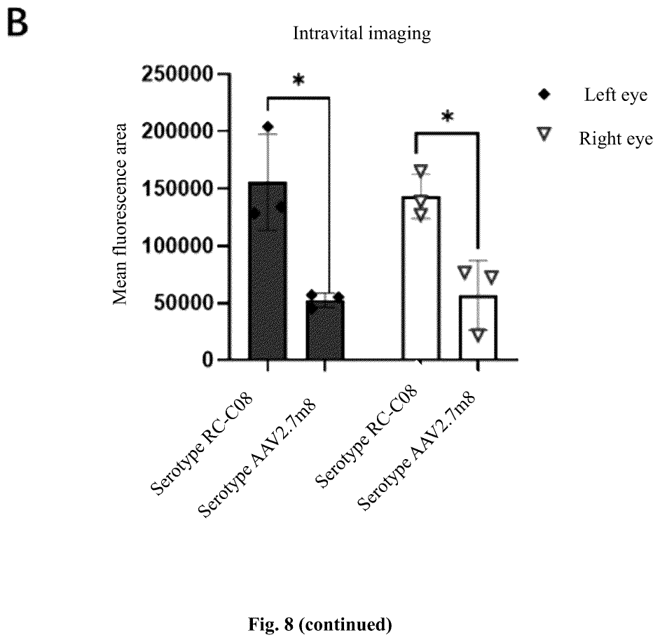

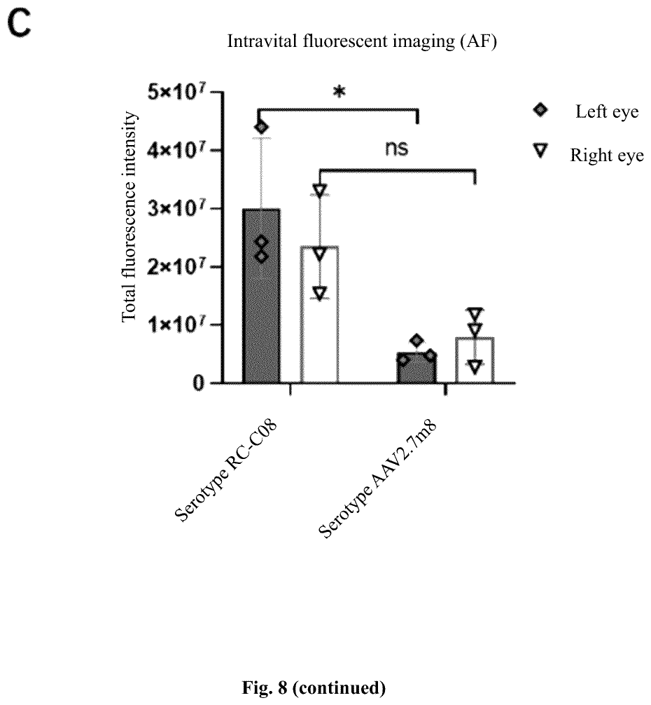

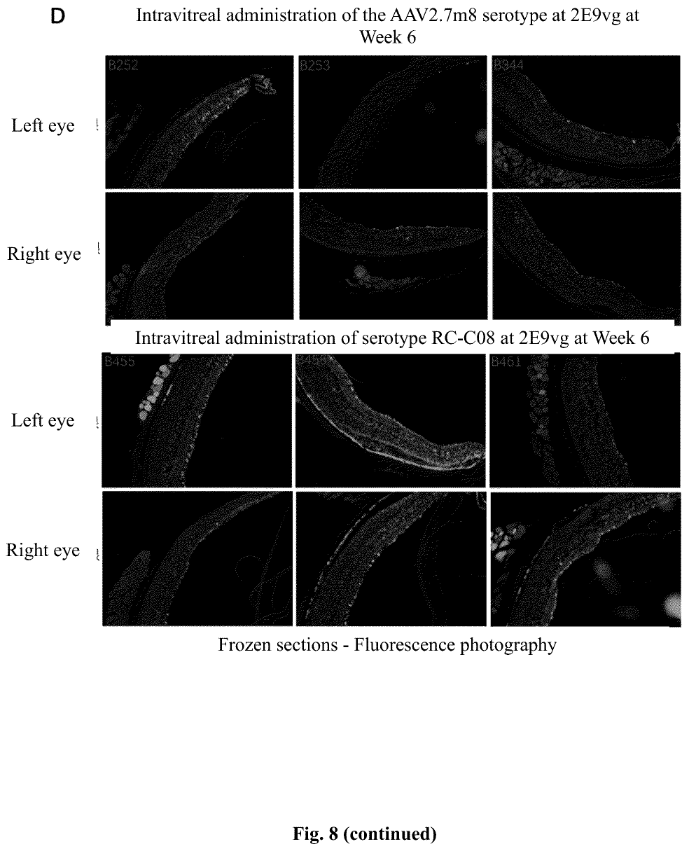

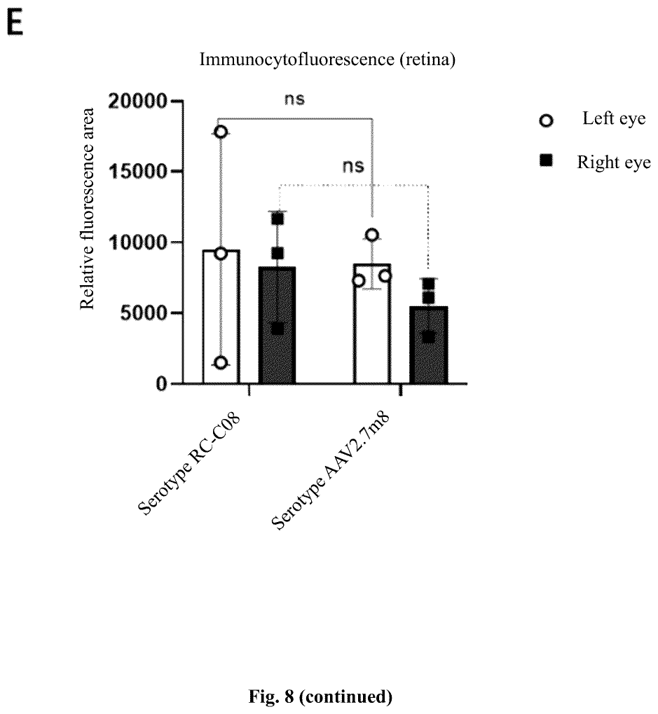

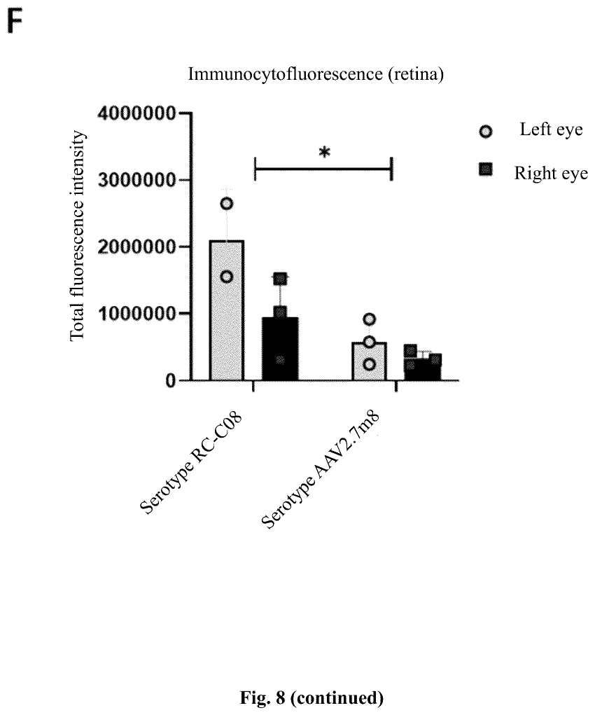

Results of auto-fluorescence assay are shown in FIGS. 8A-8C. Both of the AAV2.7m8 and RC-C08 serotypes can express the green fluorescent protein in target cells through IVT administration, thus the stronger fluorescent signals being captured through intravital fluorescence assay devices. At the same time, the in vivo transduction efficiency of the RC-C08 serotype was significantly higher than that of the control viruses, and the fluorescent signals of 6 eyes from three mice in group B were approximate, indicating the high stability of sustained expression from the RC-C08 serotype in intraocular tissues. By further analysis with ImageJ analytical software, the results of statistical analysis on the fluorescence intensity and fluorescence area (FIGS. 8B-8C) show that the relative fluorescence area of the RC-C08 serotype is more than three times that of AAV2.7m8, and the total fluorescence intensity was also increased by 3-4 times compared to the control groups.

The results of frozen sections are as shown in FIGS. 8D-8F. It is shown in FIG. 8D that, after IVT administration of the existing serotype AAV2.7m8, the tissue distribution thereof is localized. This serotype can't permeate through the retinal tissue into the RPE layer and exhibits weaker fluorescence intensity. In contrast, RC-C08 distributes throughout all of the layers in the retinal tissue of the six eyes in the administration group, with the fluorescence brightness much higher than that of the control group. The statistical data in FIGS. 8E-8F are consistent with the results of the fluorescence images.

### Example 9: Comparison of the Activity of the Serotypes RC-C08, RC-C15 and RC-C18 Tolerant to Neutralizing Antibodies of Human Origin

### Experimental Approach for Tolerance to Neutralizing Antibodies:

1. Plating: HEK-293T cells are counted with a cell counter and plated into 96-well plates with 5E3 of cells/well plated. Viral infection was performed upon the cells reaching 30%-40% confluence.
2. Diluting Virus: cells in a single well is counted before infection and the required viral volume is calculate for each well cells using the formula: V (μl)=MOI*number of cells/virus titer*1000. The virus is diluted with D□E□ complete culture medium as the diluent; Dilution of antibody (intravenous human immunoglobulin PH4, Shandong Taibang, concentration of 5%): the antibody is diluted serially 4-fold with DMEM complete culture medium as a diluent, obtaining antibody solutions with dilutions of 1 (the antibody stock solution), 1:4, 1:16, 1:64, 1:256, 1:1024, 1:4096, and 0 (in DMEM complete culture medium);
3. Co-Incubating the Virus and Antibodies: mix well the antibody and virus at a volume ratio of 1:1 and place the mixture into an incubator at 37° C. for overnight incubation;
4. Infected with the Virus: cells in a single well is counted before infection, with actual MOI calculated and documented. 100 μl of the mixed solution of virus and antibodies is added to each well, with 2 duplicate wells per each gradient for each sample. After infection the plate is placed into an incubator at 37° C. with carbon dioxide for incubation.
5. Flow Cytometry (Cytoflex, Beckman): 72 hours after infection, remove the cell supernatant and wash the cells once with PBS. After thorough digestion with trypsin, add DMEM containing 10% FBS to stop the digestion. Then, perform flow cytometry and export the results to analyze the percentage of fluorescent cells using FlowJo software.

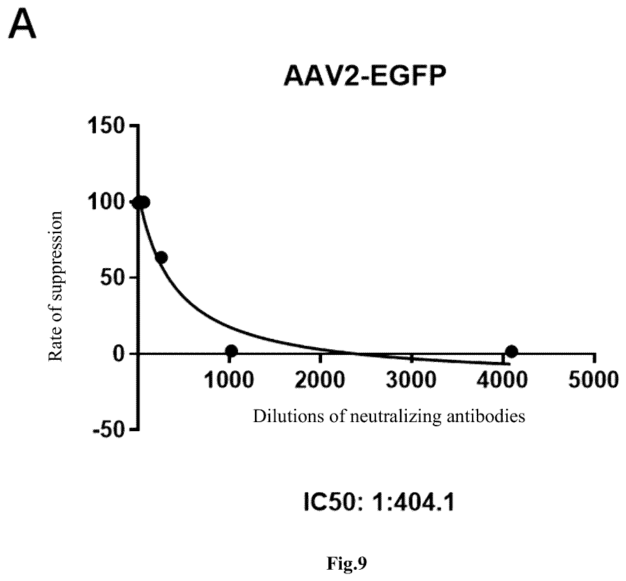

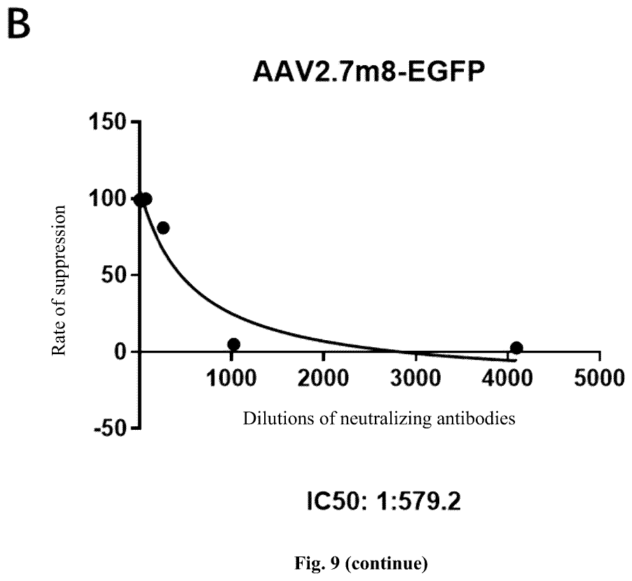

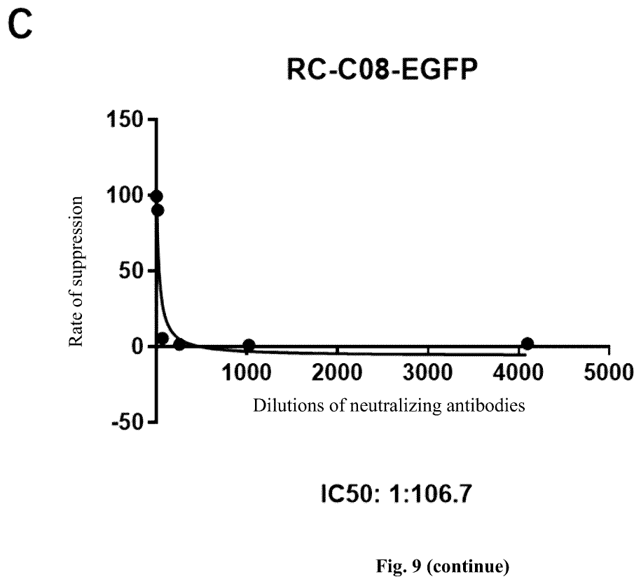

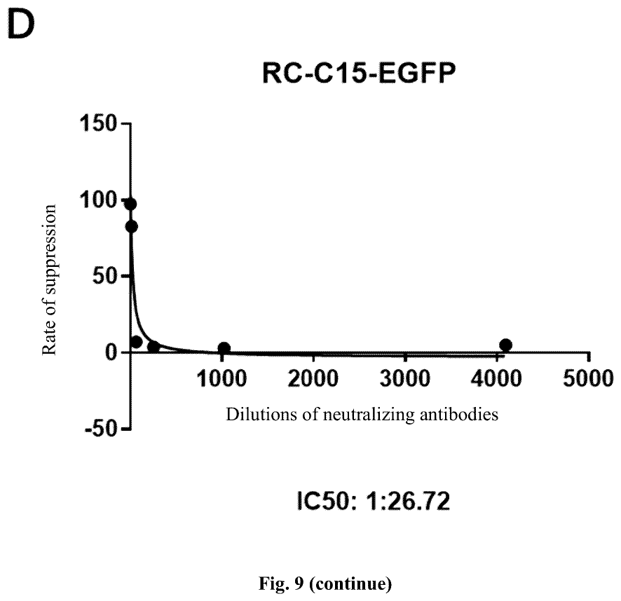

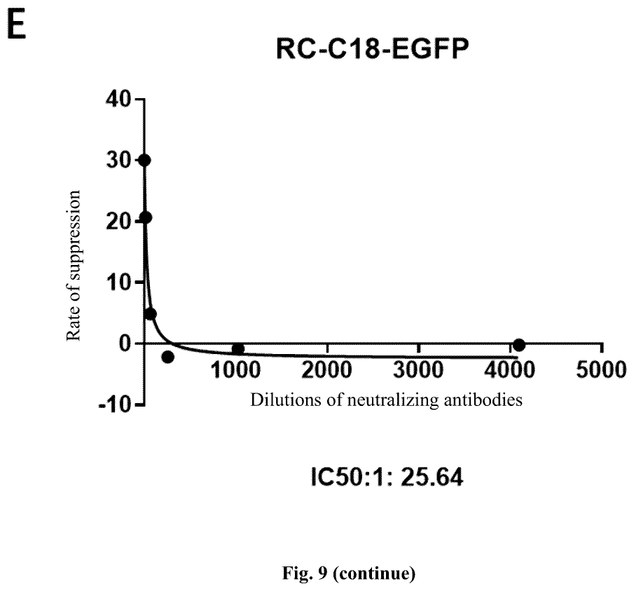

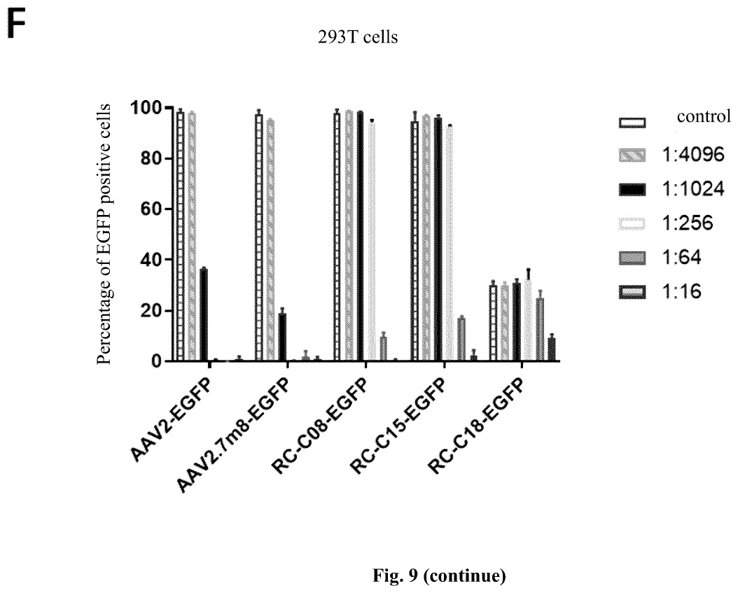

As shown in FIG. 9, a low dilution ratio represents the high amount of antibodies added, and a lower value of dilution ratio for IC₅₀ indicates that the serotype is more difficult to be neutralized by antibodies. The IC₅₀ dilution ratios corresponding to the serotypes AAV2, AAV2.7m8, RC-C08, RC-C15, and RC-C18 in 293T are 1:404.1, 1:579.2, 1:106.7, 1:26.72, and 1:25.64, respectively (see FIGS. 9A-9E). From this, it can be seen that the abilities of RC-C08, RC-C15, and RC-C18 to resist neutralizing antibodies is much higher than those of AAV2 and AAV2.7m8 known in the prior art. In the experiment, the five serotypes were ranked from strongness toward weakness as: RC-C18, RC-C15, RC-C08, AAV2, and AAV2.7m8, based on their abilities to escape from or tolerate neutralizing antibodies.

### Example 10: Comparison of the Transduction Efficiency of RC-C08 and Its Variant RC-C15 in Mouse Ocular Tissues

As shown in FIGS. 6A and 6C, which discloses the in vitro infection experiments of RC-C15 in 293T and ARPE19 cells, the statistical results showed that there was no significant increase in the in vitro transduction activity, as compared to the serotype RC-C08. Therefore, a new animal experiment was designed to evaluate the in vivo transduction efficiency and virus stability. Each serotype was administered to the left and right eyes of 3 mice at a dose of 4E8 vg, and then the intravital fluorescence assay (AF) was performed at two points in time: Week 2 and Week 4 post infection.

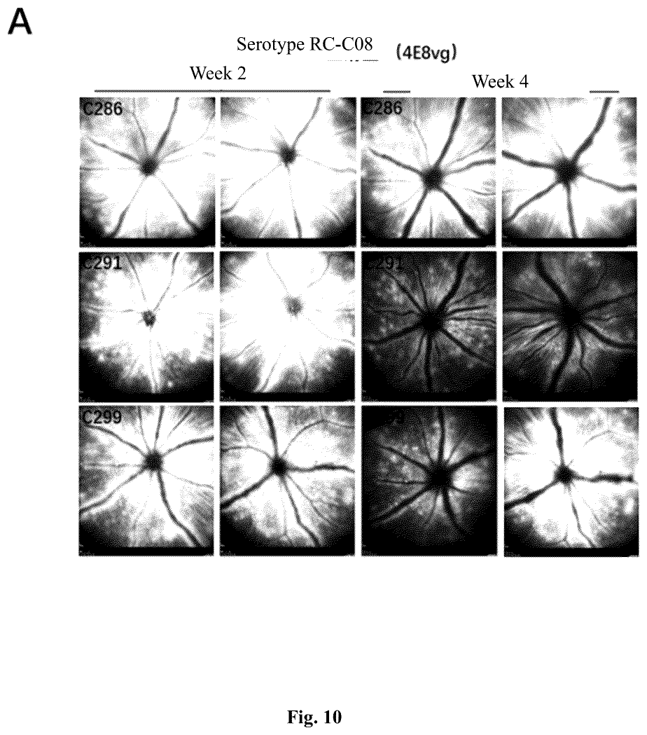

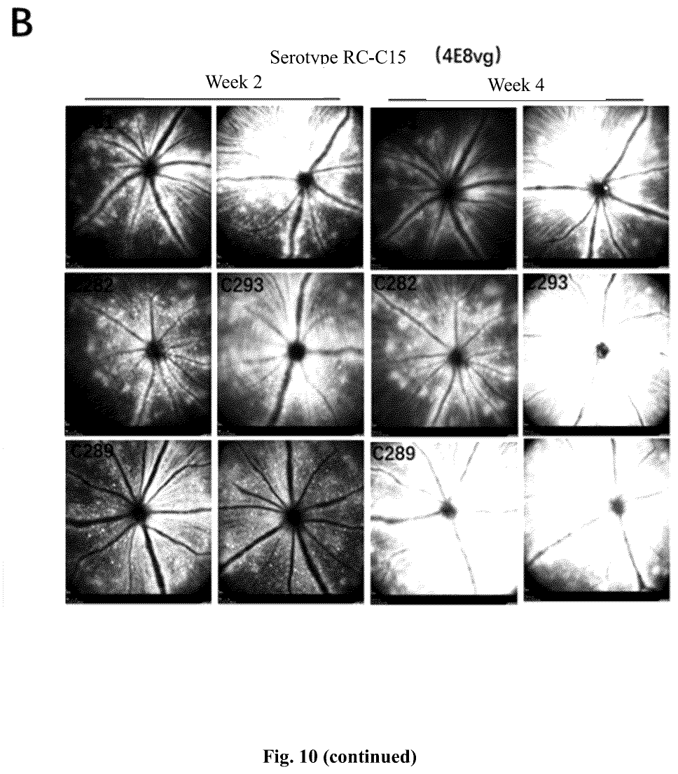

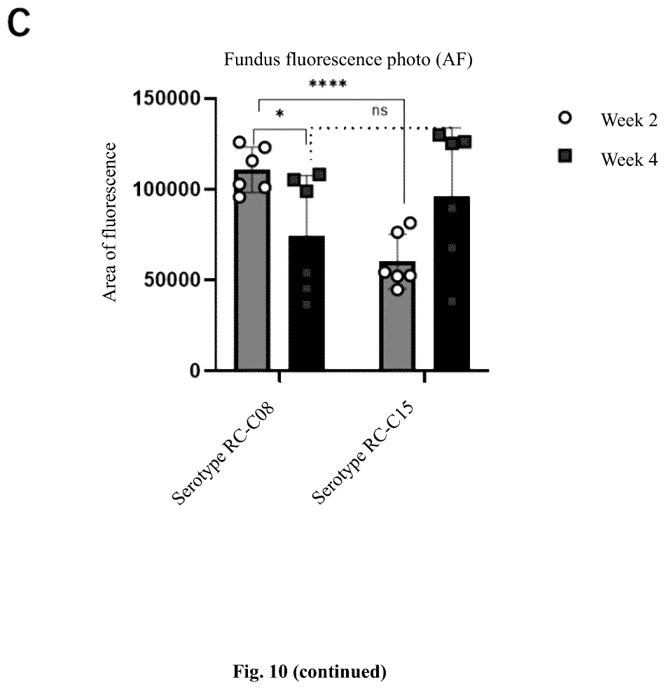

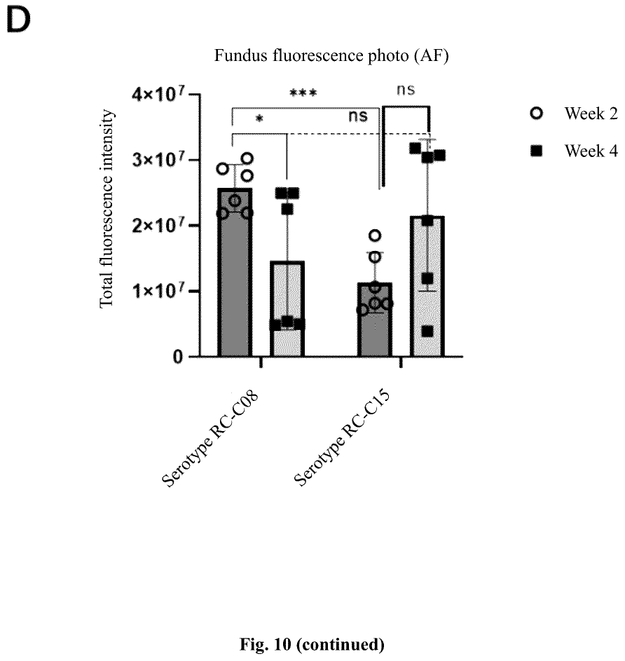

From the photos in FIG. 10A, it can be seen that 2 weeks after IVT administration, the expression intensity of the RC-C08 serotype reaches its peak, and the expression intensity in three eyes decreases significantly after 4 weeks. Similarly, the AF results as shown in FIG. 10B indicate that the RC-C15 serotype has stronger fluorescent signal in 2 weeks (weaker than that of the RC-C08 group). Over time, the intravital fluorescence photographic results at Week 4 showed that the fluorescence intensities of 3 out of 6 eyes significantly increased as compared to those at Week 2, and the values of fundus autofluorescence signal in these three eyes were stronger than those in the RC-C08 administration group at Week 4.

The total fluorescence area (FIG. 10C) and fluorescence intensity (FIG. 10D) of fundus images were further analyzed with ImagJ. Statistical results showed that the fluorescence intensity and area of the RC-C08 group were higher than those of the RC-C15 group at Week 2. Although the overall fluorescence intensity of the RC-C15 group was higher than that of the RC-C08 group at Week 4, there was no significant statistical significance due to individual differences between the groups. However, the images of the fundus autofluorescence assay intuitively displayed that the stability of expression level of GOI exhibited by the RC-C15 serotype in mouse ocular tissues was better than that exhibited by the RC-C08 administration group.

All publications, patents, and patent applications mentioned above in the specification are hereby incorporated by reference to the same extent as if each individual publication, patent, or patent application was expressly and individually indicated to be incorporated by reference in its entirety. Various modifications and variations of the method, pharmaceutical composition, and reagent kit of the present invention are readily apparent to those of skill in the art without departing from the scope and spirit of the invention. Although the invention has been described in connection with specific preferred embodiments, it should be understood that the invention can be further modified and the claimed invention should not be unduly limited to such specific embodiments. Indeed, it is apparent to those skilled in the art that various modifications on the way of implementing the present invention are intended to be within the scope of the present invention. This application is intended to encompass any variations, uses, or engineerings of the present invention which generally follow the principles of the present invention and include such deviations from the present disclosure that are in the known practices within the field to which the present invention belongs and may be applied to the basic characteristics aforementioned herein.

What is claimed is:

1. A method for engineering a recombinant adeno-associated virus vector based on adeno-associated virus serotype-2, comprising the steps of:
(a) engineering the capsid protein VP1 to comprise the following amino acid mutations: Q464V, A467P, D469N, I470□, R471A, D472V, S474G, Y500F, and S501A;
(b) engineering the capsid protein VP1 to comprise the following amino acid mutations: Q464V, A467P, D469N, I470□, R471A, D472V, S474G, Y444F, Y500F, S501A, and Y730F;
or
(c) engineering the capsid protein VP1 to have the following amino acid mutations: Q464V, A467P, D469N, I470M, R471A, D472V, S474G, Y500F, and S501A, simultaneously the amino acid sequence LALGDVTRPA inserted between 587N and 588R,
wherein the said recombinant adeno-associated virus vector comprises the said capsid protein VP1 and an exogenous gene in its genome.

2. The method of claim 1, characterized in that the step of engineering the capsid protein VP1 is achieved by modifying the nucleic acid sequence of the cap gene encoding VP1 and expressing the cap gene to obtain the VP1 protein comprising the engineered sequence.

3. The recombinant adeno-associated virus vector, obtained by the method of claim 1 or 2.

4. An isolated VP1 capsid protein, characterized by comprising the amino acid sequence set forth in any one of SEQ ID NOs: 1-3.

5. A nucleic acid molecule, encoding the VP1 capsid protein of the recombinant adeno-associated virus vector of claim 3 or the VP1 capsid protein of claim 4.

6. The nucleic acid molecule of claim 5, comprising a nucleic acid sequence set forth in or having 70% sequence identity with any one of SEQ ID NOs: 4-6.

7. A recombinant adeno-associated virus vector, comprising:
(i) a VP1 capsid protein of claim 4; and
(ii) an exogenous gene, which can be expressed after infection.

8. The recombinant adeno-associated virus vector of claim 7, characterized in that the exogenous gene of (ii) encodes a therapeutic protein.

9. The recombinant adeno-associated virus vector of claim 7, characterized in that the exogenous gene of (ii) is a reporter gene.

10. The recombinant adeno-associated virus vector of claim 9, characterized in that the exogenous gene of (ii) is a green fluorescent protein gene.

11. A pharmaceutical composition, comprising the recombinant adeno-associated virus vector of claim 3, 7 or 8.

12. The pharmaceutical composition of claim 11, characterized in that the pharmaceutical composition is administered by a route of systemic or topical administration.

13. The pharmaceutical composition of claim 11, characterized in that the pharmaceutical composition is administered by a route of is intravenous, intra-muscular, subcutaneous, oral, topically contact, intraperitoneal, or intralesional administration.

14. The pharmaceutical composition of claim 9, characterized in that the pharmaceutical composition is administered by a route of eye drop instillation, intra-ocular, conjunctival, intracameral, intravitreal, or subretinal injection.

15. Use of the recombinant adeno-associated virus vector of claim 3, 7, or 8, or the pharmaceutical composition of any one of claims 11-14 in the manufacture of a medicament for treating a disease.

16. The use of claim 15, wherein said disease is an ocular disorder.

17. The use of claim 15, wherein said disease is a retinal disease, for example, IRD.

18. The use of claim 15, wherein the medicament is administered by a route of intravenous, intra-muscular, subcutaneous, oral, topically contact, intraperitoneal, or intralesional administration.

19. The use of claim 15, wherein the medicament is administered by a route of eye drop instillation, intra-ocular, subconjunctival, intracameral, intravitreal, or subretinal injection.

20. The use of claim 15, wherein said medicament is used for the treatment of an individual who had been treated with rAAV vectors and/or naturally infected by AAV.

21. A host cell, comprising the nucleic acid molecule of claim 5 or 6.

22. A host cell, comprising the recombinant adeno-associated virus vector claim 3, 7, or 8.

23. The host cell of claim 21, further comprising one or more of additional vectors for packaging adeno-associated viruses.

24. A method for producing a recombinant adeno-associated virus vector capable of expressing an exogenous gene sequence, the method comprising the steps of:
(i) introducing the following into cells:
(a) the nucleic acid molecule of claim 5 or 6,
(b) a vector harboring the exogenous gene sequence, and
(c) one or more of the additional recombinant vectors for packaging adeno-associated viruses;
then
(ii) expressing viral proteins encoded by said nucleic acid molecule and said vector for packaging adeno-associated virus in said cells, wherein the viral proteins enclose the vector harboring the exogenous gene sequence to form viral particles, therefore giving rise to a recombinant adeno-associated virus containing the exogenous gene sequence;
optionally
(iii) collecting the recombinant adeno-associated virus.

25. The method of claim 24, wherein said cells are HEK-293 cells or derived from HEK-293 cells and the said cells are grown adherently or in suspension.


SEQUENCE LISTING (13 sequences)

SEQ ID NO: 1 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSVAGPSNMAVQGRNWLPGPCYRQQRVSKTSADNNNSEFAWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSVAGPSNMAVQGRNWLPGPCYRQQRVSKTSADNNNSEFAWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 3 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSVAGPSNMAVQGRNWLPGPCYRQQRVSKTSADNNNSEFAWTGATKYHLNGRDSLVNPGPAMASHKDDEEKFFPQSGVLIFGKQGSEKTNVDIEKVMITDEEEIRTTNPVATEQYGSVSTNLQRGNLALGDVTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRFLTRNL

SEQ ID NO: 4 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctgtggccggaccgagtaacatggcggtccagggtaggaactggcttcctggaccctgttaccgccagcagcgagtatcaaagacatctgcggataacaacaacagtgaattcgcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 5 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttgagcagaacaaacactccaagtggaaccaccacgcagtcaaggcttcagttttctgtggccggaccgagtaacatggcggtccagggtaggaactggcttcctggaccctgttaccgccagcagcgagtatcaaagacatctgcggataacaacaacagtgaattcgcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagattcctgactcgtaatctgtaa

SEQ ID NO: 6 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctgtggccggaccgagtaacatggcggtccagggtaggaactggcttcctggaccctgttaccgccagcagcgagtatcaaagacatctgcggataacaacaacagtgaattcgcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgacgtaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagattcctgactcgtaatctgtaa

SEQ ID NO: 7 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 735 aa, synthetic construct [sequence omitted]

SEQ ID NO: 9 - DNA, 25 bp, synthetic construct
  source 1..25: mol_type = other DNA
gacgtcagacgcggaagcttcgatc

SEQ ID NO: 10 - DNA, 24 bp, synthetic construct
  source 1..24: mol_type = other DNA
gctgtttaaacgcccgggctgtag

SEQ ID NO: 11 - DNA, 18 bp, synthetic construct
  source 1..18: mol_type = other DNA
cacaatcttggctcactg

SEQ ID NO: 12 - DNA, 18 bp, synthetic construct
  source 1..18: mol_type = other DNA
ctggaatcccaacaactc

SEQ ID NO: 13 - DNA, 21 bp, synthetic construct
  source 1..21: mol_type = other DNA
ttcaagcgattctcctgcctc